(19) United States
(12) Patent Application Publication
(10) Pub. No.: US 2009/0310839 A1
(43) Pub. Date: Dec. 17, 2009

(54) SYSTEMS AND METHODS TO PERFORM INHIBITION DIAGNOSTIC TESTING

(76) Inventors: Omer Y. Katzenelson, Encinitas, CA (US); Ileana L. Katzenelson, Encinitas, CA (US)

Correspondence Address: BLACK LOWE & GRAHAM, PLLC, 701 FIFTH AVENUE, SUITE 4800, SEATTLE, WA, 98104, US

(21) Appl. No.: 12/483,199

(22) Filed: Jun. 11, 2009

Related U.S. Application Data
(60) Provisional application No. 61/060,727, filed on Jun. 11, 2008.

(51) Int. Cl. G06K 9/00; C12Q 1/02

(52) U.S. Cl. 382/128; 435/29

(57) ABSTRACT

System and methods to perform inhibition diagnostic testing utilizing a Petri dish based packaged applied materials kit to conduct growth inhibition testing of essential oil compositions, herbs, antibiotics, antifungals, anti-parasititic compounds applied to fungi, bacteria or other biological materials having confluent growth patterns that are derived from a patient's inoculum grown in a personal incubator. Resulting inhibition patterns are generated by at least one essential oil and/or a panel of essential oils or other applied materials distributed in fixed concentrations or concentration gradients to confluent lawns of bacterial and/or fungal lawns derived from the patient's clinical specimen. Images are acquired during the incubation periods to measure inhibition areas or zones that developed around applied the applied material compositions and the optimal therapeutic treatment is ascertained from analysis of the developing or generated inhibition zones.

### CROSS REFERENCES TO RELATED APPLICATIONS

This application claims priority to and incorporates by reference in its entirety U.S. Provisional Patent application Ser. No. 61/060,727 filed Jun. 11, 2008.

### FIELD OF THE INVENTION

An embodiment of the present invention relates to systems and methods to perform aromatogram and other forms of inhibition diagnostic testing from a packaged test kit.

### BACKGROUND OF THE INVENTION

Some antibiotics have become therapeutically ineffective to treat a number of bacterial and fungal conditions, and antibiotic testing in clinical settings using specialized laboratories often is inconvenient to patients. Often a patient has to undergo random antibiotic testing and endure the possibility of being confronted with negative reports that the antibiotics being tested for a given bacterial or fungal infection are ineffective. The negative results are even harder for the patient to endure when the patient has been taken antibiotics that are found to be ineffective yet has also disrupted the normal and good intestinal bacterial flora and possibly compromised the patient's immunity. In broad spectrum antibiotics, it is possible that the normal intestinal bacterial fauna is obliterated, resulting in an imbalance of Clostridium difficile populations. Excessive C. difficle levels can exhibit toxic effects, especially when the very elderly and pediatric populations are overwhelmed with Toxin A or Toxin B producing C. difficle strains. Currently society is burdened by the growing incidences of antibiotic resistant bacteria found to be flourishing in hospitals, medical offices and schools to which antibiotic therapy has been ineffective.

### SUMMARY OF THE INVENTION

System and methods are described to perform inhibition diagnostic testing and to dispose medical waste therefrom. The inhibition diagnostic testing includes essential oil aromatograms, antibiotic disk diffusion, antifungal disk diffusion, herbal inhibition, and cell inhibition studies. The various forms of the inhibition diagnostic testing may be performed in a non-laboratory setting, including the domicile or personal residence of a patient user. The systems and methods utilizes a Petri dish based packaged inhibition kit and incubator. In the case of essential oils, growth inhibition derived from the patient's own biological specimens include aromatogram testing of essential oil compositions applied to fungi, bacteria or other biological materials having a confluent growth pattern derived from the patient's inoculums.

The patient's inoculums are grown in a personal incubator at time, temperature, and other condition (pH, nutrients, gas flow) having settings that are optimal for growing the applied inoculum. Resulting aromatograms are generated by a panel of essential oils applied in fixed concentrations or concentration gradients to confluent lawns of bacterial and/or fungal lawns derived from the patient's clinical specimen. Images are acquired during the incubation periods and image analysis of the developing or developed aromatograms are undertaken using image analysis software having instructions to perform pattern recognition of the developing and/or generated aromotogram's inhibition zones. From the analysis of the developing or generated inhibition zones, the patient can predict which oil concentration and/or oil combination may provide the optimal therapeutic treatment.

### BRIEF DESCRIPTION OF THE DRAWINGS

Illustrative and alternative embodiments of the present invention are described in detail below with reference to the following drawings.

FIG. 1 is a schematic overview of an Inhibition Testing System;

FIG. 2 depicts a flowchart for an Inhibition Diagnostic Procedure;

FIG. 3 depicts a method to develop a gradient streak;

FIG. 4 schematically depicts an alternate method to develop a gradient streak or applied discrete spots with self-registration of the applied materials 32;

FIG. 6 depicts a series of one-dimensional fabric-based gradient strips;

FIGS. 6A-B 4 schematically depict perspective views of an ambient temperature Personal Incubator;

FIGS. 7A-C schematically depict perspective views of a temperature controlled and sanitizable personal incubator; and

FIG. 8 depicts an alternate embodiment of the personal incubator of FIGS. 6A-C having a tiltable base.

### DETAILED DESCRIPTION OF THE PARTICULAR EMBODIMENTS

Described herein include system and methods to perform inhibition diagnostic testing utilizing a Petri dish based packaged applied materials kit to conduct growth inhibition testing of essential oil compositions, herbs, antibiotics, antifungals, anti-parasititic compounds applied to fungi, bacteria or other biological materials having confluent growth patterns that are derived from a patient's inoculum grown in a personal incubator. Resulting inhibition patterns are generated by at least one essential oil and/or a panel of essential oils or other applied materials distributed in fixed concentrations or concentration gradients to confluent lawns of bacterial and/or fungal lawns derived from the patient's clinical specimen. Images are acquired during the incubation periods to measure inhibition areas or zones that developed around applied the applied material compositions and the optimal therapeutic treatment is ascertained from analysis of the developing or generated inhibition zones.

Figures described below illustrate particular embodiments for systems and methods to perform aromatogram essential oil, antibiotics, antifungals, and herbal inhibition diagnostic testing against a patient's biological sample grown to confluency on an agar or other growth media plate. The methods include diagnostic procedures including a patient questionnaire to guide the patient to select a region of the patient's body to obtain a specimen to grow to confluency, how to grow the patient's biological specimen to attain a confluent lawn on an agar surface, how to operate a personal incubator of various designs to assist in developing agar plate confluency, how to perform inhition testing, how to acquire and analyze images of the inhibition zones, and to describe the protocol to report the identification to a central collection center.

The figures also illustrate particular embodiments for systems and methods to perform a safe, outside a laboratory setting, an aromatogram diagnostics. An aromatogram diagnosis is the in vitro killing/inhibition of a pathogen. A correlation between in vitro activity and in vivo activity was established in numerous cases and is still been investigated. See for example reference: BMC Microbiology 2009, 9:88, Invest Ophthalmol Vis Sci 28:596-603, 1987, herein incorporated by reference in its entirety. In other words, from the analysis of the developing or generated inhibition zones, the patient or at home user can predict which medication (essential oil, traditional Chinese Medicine, etc) may provide optimal therapeutic treatment.

Note that there might be a serious implication of using or avoiding specific medications due to drug interaction if the person or patient is taking another drug at the same time or has other medically related conditions.

1, a life threatening situation exists and necessitates that immediate medical assistance be immediately sought, and

2, a questionnaire is completed about a patient's overall health, current conditions and symptoms, and a certain result in indicated for a set of conditions and steps to follow. The steps include, for example, which body part or regions to swab, the media type to use, and whether any specific temperature, duration, or other special condition (i.e., an external gas flow) is required. Additionally, disposal instructions of the swab and Petri dish may be indicated.

In the beginning the patient may consult their professional medical practitioner regarding the questionnaire, and obtain the practitioner's input concerning the suspected pathogen, a medical diagnosis & treatment guide book, or software to utilize. For example, the “Current Medical Diagnosis and Treatment 2008” by McPhee at el. For each suspected pathogen consult American Type Culture Collection (ATCC) (or “Laboratory Diagnosis of Infectious Diseases: Essentials of Diagnostic Microbiology”2007 by Engelkirk at el.) for testing procedure (test duration, temperature, gas flow etc), herein incorporated by reference in its entirety.

In another scenario, should the patient suspect a yeast infection the practitioner may guide him to collect sterile midstream urine. Here are few possible conditions:1)cystine-lactose-electrolyte-deficient agar plate and incubated at 37° C. in an atmosphere containing 6% CO₂ for one day or 2) sheep blood agar or 3) MacConkey agar plates for one day, Or 4) Schaedler agar for two days, etc. Media and media incubation conditions may be obtained from Tena D, González-Praetorius A, Sáez-Nieto J A, Valdezate S, Bisquert J. Urinary tract infection caused by capnophilic Escherichia coli Emerg Infect Dis 2008, herein incorporated by reference in its entirety.

The software will record the practitioner recommendation, the protocol you used and your feedback (was the test relevant and did it cure or ease the symptom). Later, once enough conditions and feedback were recorded the software will provide recommendation for an appropriate test. Note that each practitioner may lend toward medicine common in his practice depending or where trained. For example a Western-trained practitioner may favor more antibiotics, a traditional Chinese practitioner will tend to favor more Chinese herbs, and a European trained practitioner will tend to favor more essential oils, hydrosols and tinctures. They all can test their medicine using the system and methods described below.

1. Prepare media or use a prepared petri dish according to the result of step #2

2. Collect sample with a sterile swab.

3. Apply swab inoculums on the Petri dish surface. The Petri dish surfaces may be wafted by a flame applied approximately about 5 cm from the open flame. FIG. 2 below provides additional details for an inhibition diagnostic test procedure from the swab specimen.

4. Close petri dish until sample is dry.

5. Disinfect incubator—rinse with antibacterial/anti fungal rinse or expose for few minutes to UV lamp.

6. Install a new filter into the incubator.

7. Remove dish cove and place dish up side down inside the incubator (placing them up side down ensure that they will not be contaminated by dust particles that mat fall on them)

8. Close incubator.

9. Set up incubation conditions (time, temperature, extra gas flow if needed)

10. Monitor or measure inhibition zone (larger zone correlates to better inhibition).

11. Report results

12. Disinfect the incubator (apply UV lamp or rinse with disinfectant and drain the liquids in both cases allow for few minutes for the microbe to be destroyed), and

13. Dispose, or sterilize the Petri dish for reuse.

up of inoculation media, inoculums, and essential oil drops, disks, and/or gradient strips.

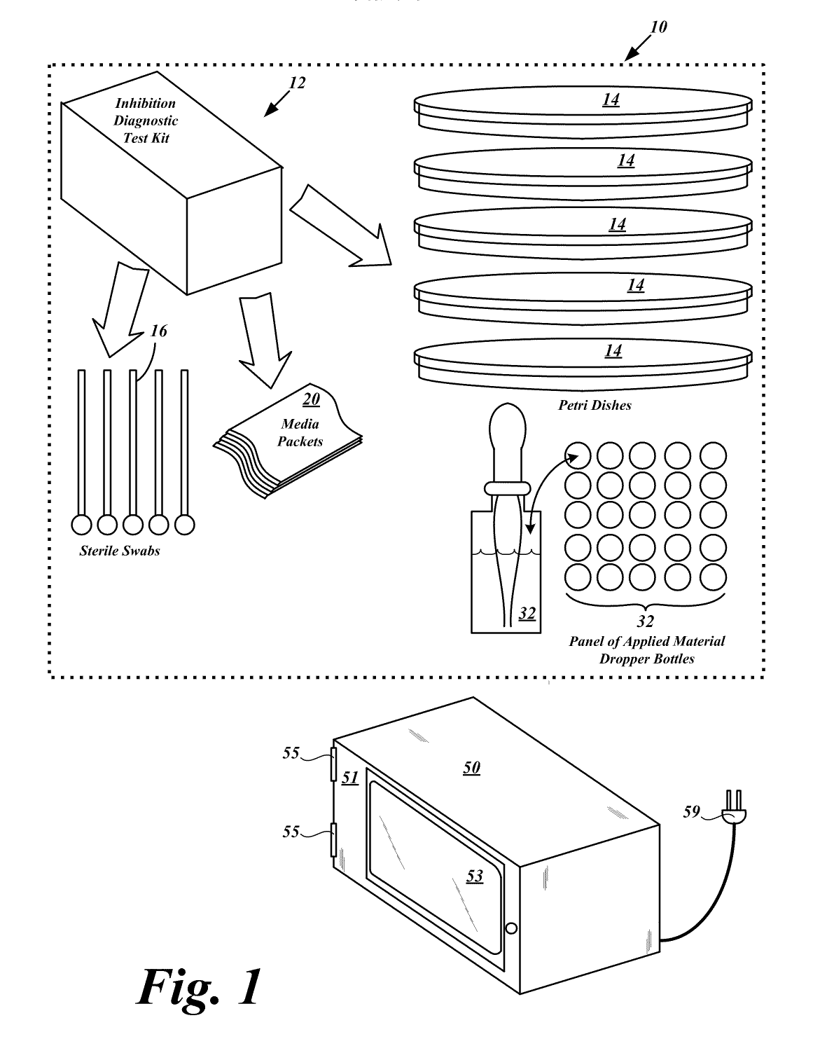

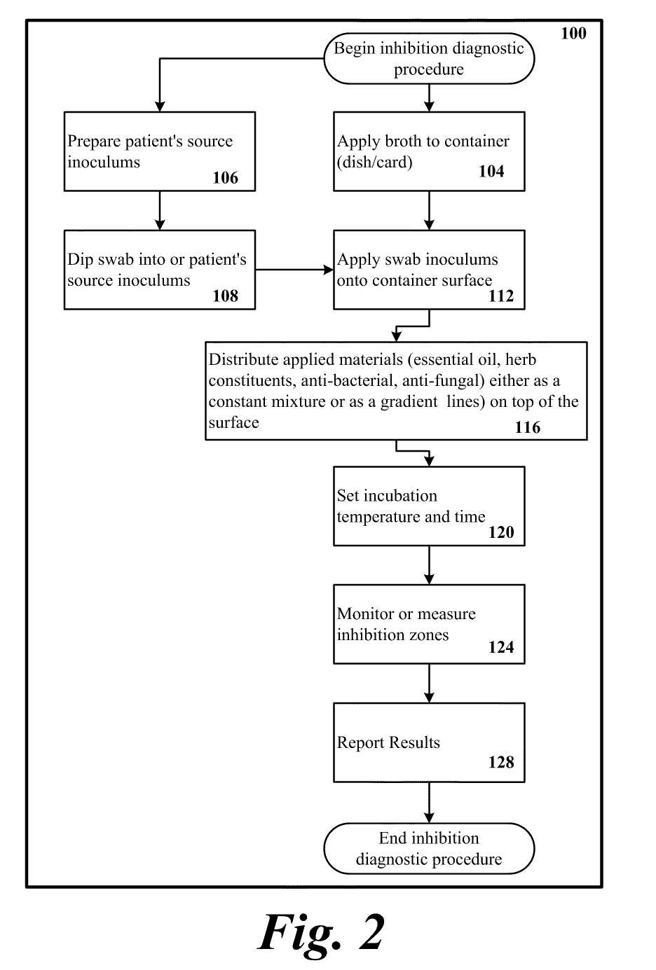

FIG. 1 is a schematic overview of an Inhibition Diagnostic Testing System 10. In general the system 10 provides for the aromatogram diagnostic testing system includes an aromatogram or applied materials kit 12 and an incubator 50. The applied materials kit 12 includes at least one Petri dish 14, a Petri dish lid 15, at least one sterile swab 16, at least one media packe 20t, and at least one applied material 32 example include but not limited to any one component or a mixture of the following: essential oil composition, hydrosol, tincture, Chinese herbs, silver ions, antibiotics or nano sized emulations of oil in water.

Other embodiment may include a Petri dish 14 with growth media, for example, a composition of agar, already in the Petri dish 14.

Other embodiment will have the media with an inhibitor. In some cases the media will contain substance(s) to inhibit the growth of unwanted bacteria or fungus. An inhibitor may be used after the user establishes his goal to test for specific microbe. If we are testing for bacteria an anti-fungal solution will be used as the inhibitor, and vice versa. This substance is called inhibitor, and this term will be used hereon. For example any traditional antibiotic can also be used. For bacterial growth, anti fungal material will be supplied and for fungal growth anti bacterial will be supplied, as disclosed in U.S. Pat. No. 5,403,587 by McCue, herein incorporated by reference in its entirety.

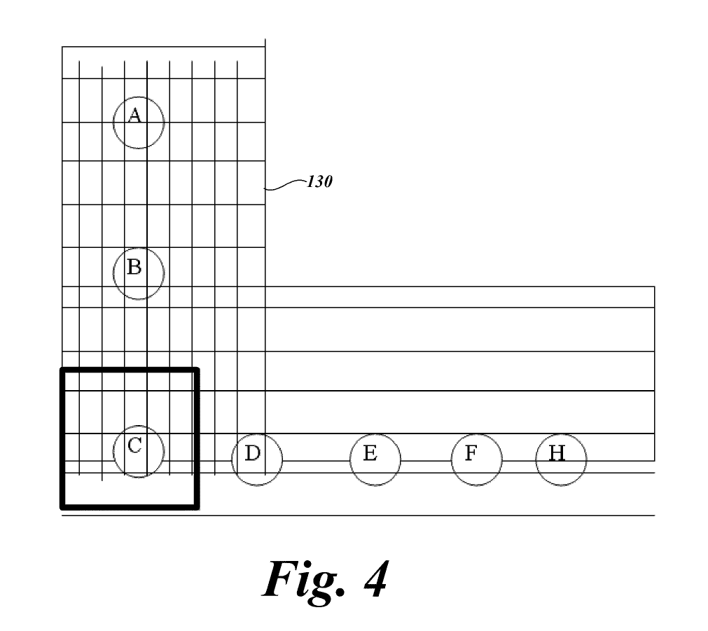

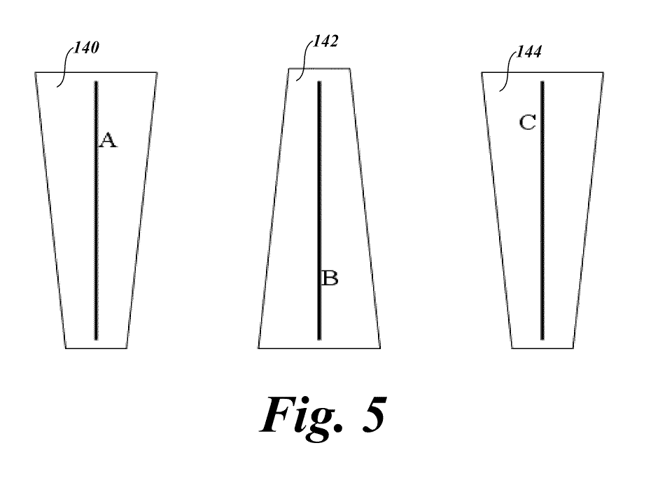

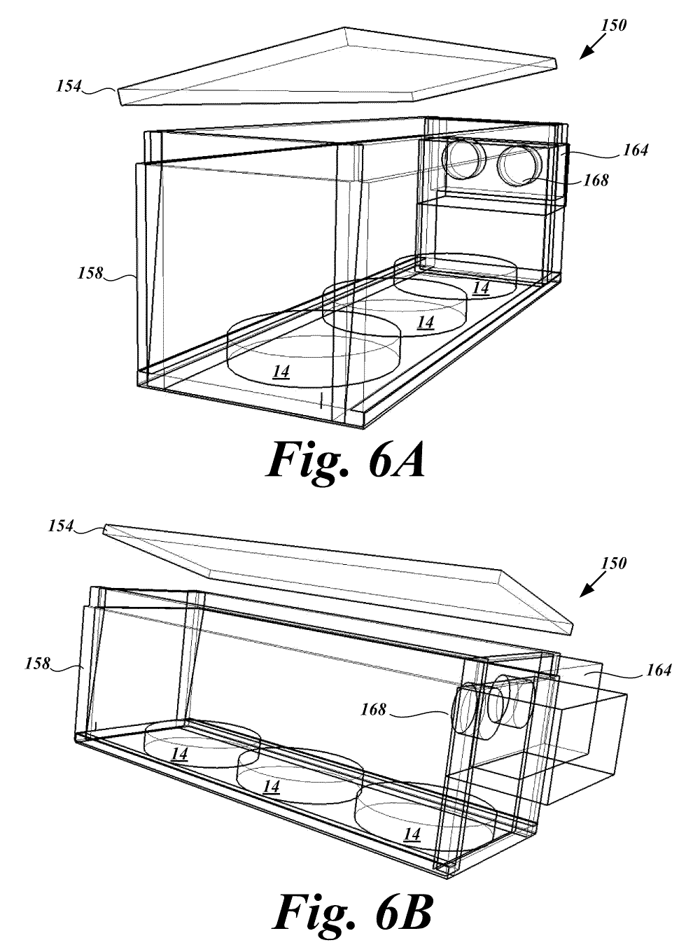

FIG. 2 depicts a flowchart for an Inhibition Diagnostic Procedure. The Inhibition Diagnostic Procedure or method 100 of using the Inhibition Testing System 10. The method 100 begins with two parallel paths that converge. One path includes preparing the patient's source inoculum or inoculums at process block 106, followed by dipping the sterile swab 16 into the prepared patient's inoculum or inoculums with a separate dedicate swab 16 per each inoculum. The other path includes applying broth to the Petri dish 14. After preparation, at process block 112 where the parallel paths converge, the inoculated swab 16 is applied to the prepared Petri dish 14 to make a patient source confluent lawn. Thereafter, at process block 116, applied materials 32 are applied in an array to the human source inoculated plates, either as undiluted drops, or drops diluted with diluents to provide a gradient streak (as shown in FIGS. 4 and 5 below), or applied as disks Then, at process block 120, the oil-applied plates are placed in the incubator 50 for a defined temperature and incubation period. At process block 124, the kill zones developing around the applied oil drops and/or gradient streaks are observed. Optionally, the kill zones may be observed by the digital camera 54 and images acquired. Pattern recognition analysis is performed on the kill zones or inhibitory areas to the acquired images. Pattern recognition software instruction can detect the presence of inhibition and characterize the progress of a given essential oil composition's inhibition. At process block 128, results of the inhibition diagnostic testing procedure are reported to complete the method 100. The method 100 encompasses media, specimen growth, and applied materials 32. The method may be modified for agar plates 14 serving as a negative control, that is, receiving media only to confirm sterility or aseptic handling techniques, and a positive control encompassing media and specimen, but not the applied materials 32, so that a dedicated plate demonstrably shows a confluent microbial lawn in the absence of applied materials 32. In the incubators 150 and 200 shown respectively in FIGS. 6A-B and 7A-C below are three Petri dishes 14. The first Petri dish 14 serves as a negative or media control, the second Petri dish 14 serves as a positive or confluent lawn control, and the third Petri dish 14 serves as the test dish having media, inoculated specimen, and applied material 32.

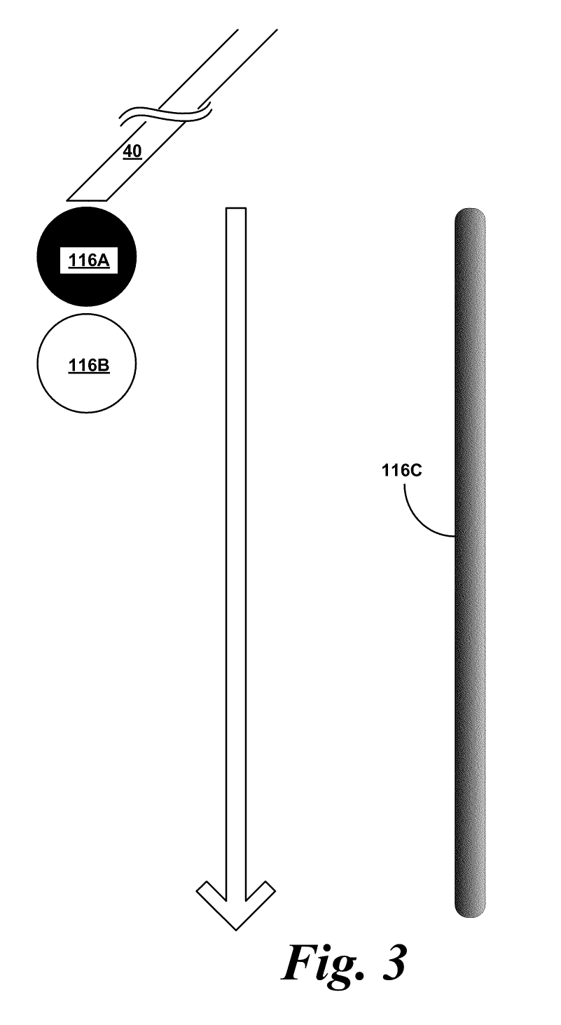

The aromatogram or inhibition diagnostic procedure described above may include procedural plate controls that accompany the essential oil test plates. The test plate controls may include a negative control derived from the sterile inoculation media obtained from the media packets 20, and a positive control representing the confirmed growth of confluent bacteria or fungal lawns grown on the agar plate. The negative control plate includes a confirmation that reconstituted media packets are sterile and is made by streaking the liquid form of the media packet onto a separate agar plate. The positive control is the liquid form of the media packet with the human sampled source inoculums streaked onto its dedicated plate in a manner designed to obtain a confluent bacterial and/or fungal lawn. The media negative control and the confluent human inoculums positive control is co-incubated with the essential oil test plate described for FIGS. 3 and 4 schematically depicted as the three agar plates 14. Alternatively, the agar plates 14 may be segmented or split with three discrete zones. One zone functioning as the negative media control, the second zone functioning as the human inoculums positive control, and the third zone serving as the applied material test zone made

Example of antibacterials for the applied materials 32 include Amdinocillin (Mecillinam), Amikacin, Amoxicillin, Amoxicillin+Clavulanate, Ampicillin, Ampicillin+Sulbactam, Atovaquone, Azithromycin, Aztreonam, Bacampicillin, Bacitracin, Capreomycin, Carbenicillin indanyl sodium, Cefaclor, Cefadroxil, Cefamandole, Cefazolin, Cefdinir, Cefditoren, Cefepime, Cefixime, Cefmetazole, Cefonicid, Cefoperazone, Cefotaxime, Cefotetan, Cefoxitin, Cefpodoxime Proxetil, Cefprozil, Ceftazidime, Ceftibuten, Ceftizoxime, Ceftriaxone, Cefuroxime and Cefuroxime axetil, Cephalexin, Cephalothin, Cephapirin, Cephradine, Chloramphenicol, Cinoxacin, Ciprofloxacin, Clarithromycin, Clindamycin, Cloxacillin, Colistimethate, Cycloserine, Daptomycin, Demeclocycline, Dicloxacillin, Dirithromycin, Doripenem, Doxycycline, Enoxacin, Ertapenem, Erythromycin, Fosfomycin, Gatifloxacin, Gemifloxacin, Gentamicin, Grepafloxacin, Imipenem/Cilastatin, Imiquimod, Kanamycin, Levofloxacin,Lincomycin, Linezolid, Lomefloxacin, Loracarbef, Mafenide, Malathion, Meropenem, Methacycline, Methenamine hippurate, Methicillin, Metronidazole, Mezlocillin, Minocycline, Moxifloxacin, Mupirocin, Nafcillin, Nalidixic Acid, Neomycin, Netilmicin, Nitrofurantoin, Nitrofurazone, Norfloxacin, Novobiocin, Ofloxacin, Oxacillin, Oxytetracycline, Penicillin, Piperacillin, Piperacillin+Tazobactam, Podofilox, Polymyxin B, Quinupristin+Dalfopristin, Retapamulin, Rifapentine, Rifaximin, Saturated Solution of Potassium Iodide (SSKI), Sparfloxacin, Spectinomycin, Streptomycin, Sulfadiazine, Sulfamethoxazole, Sulfisoxazole, Sulphur, precipitated in petrolatum, TCA (trichloroacetic acid), BCA (bichloroacetic acid), Teicoplanin, Telithromycin, Terbinafine, Tetracycline, Ticarcillin, Ticarcillin+Clavulanic Acid, Tigecyclinel Tobramycin, Trimethoprim, Trimethoprim+Sulfamethoxazole, Trovafloxacin and Vancomycin.

Examples of antifungals include Amphotericin B, Amphotericin B cholesteryl sulfate complex (ABCD), Amphotericin B lipid complex (ABLC), Amphotericin B liposomal, Anidulafungin, Caspofungin acetate, Clotrimazole, Fluconazole, Flucytosine, Griseofulvin, traconazole, Ketoconazole, Micafungin, Miconazole, Nystatin, Pentamidine, Posaconazole, Terbinafine and Voriconazole.

Antimycobacterial include Aminosalicylic Acid, Capreomycin, Clofazimine, Cycloserine, Ethambutol, Ethionamide, Isoniazid, Pyrazinamide, Rifabutin, Rifampin, Rifapentine and Streptomycin.

Examples of antiparasitic compounds include Albendazole, Artesunate, Atovaquone+Proguanil, Bephenium hydroxynaphthoate, Chloroquine, Dapsone, Diethyl-carbamazine, Diloxanide furoate, Eflornithine, Emetine Hcl, Furazolidone, Ivermectin, Lindane, Mebendazole, Mefloquine, Melarsoprol, Miltefosine, Niclosamide, Nifurtimox, Nitazoxanide, Oxamniquine, Paromomycin, Permethrin, Piperazine, Praziquantel, Primaquine, Pyrantel pamoate, Pyrimethamine, Pyrimethamine+sulfadoxine, Quinacrine Hcl, Quinidine, Quinine, Sodium Stibogluconate, Spiramycin, Thiabendazole and Tinidazole.

Other embodiment will be the media with a color indicator. It may be used to identify the family of the microbe. The color indicator technology is disclosed in U.S. Pat. No. 4,248,597 by McNeely.

Other embodiments may include media with inhibitors and at least one color indicator.

The applied material kit includes a set of sterile swabs 16, a set of media packets 20, and a panel of applied materials, Dropper bottles 32. Media packets 20 contain media formulations amenable for growing the pathogenic bacteria, fungi, parasite or cell infectious agents common to human, animal or plant infections that may be traceable to or derived from strains stored by the American Type Culture Collection (ATCC) or other bacterial and fungal repositories and growable on agar or other nutrient media media, which may include any type of broth or sugar based nutrient located in Petri dishes 14. The media packets 20 include formulations listed by the National Committee for Clinical Laboratory Standards, herein incorporated by reference.

The panel of applied material Dropper bottles 32 may include essential oils contained in a natural extract having an oil base, water base, alcohol base, Chinese herbs, silver ions, western antibiotic (see paragraph 0020), or nano size emulation of oil in water. One of the bottles may include the diluent base used to dissolve the applied material to serve as the diluent drop 116B described in FIG. 3 below for the preparation of applied material gradient streaks 116C having a declining applied material concentration range. The media packets 20 may either be in a sterile liquid formulated with constituents optimized at concentrations to grow microbes commonly found in human inoculums. Alternatively, the media packets 20 may be in sterile, dry powder form (if so they will need to be cooked before usage), in which case they will need to be cooked before using and be reconstituted aseptically using sterile water-for-injection (WFI) or sterile saline as so indicated in the reconstitution instructions listed with the media packets 20.

Alternate embodiments for the Inhibition Diagnostic Test Kit 12 may include bottles of sterile WFI and/or sterile saline when necessary for reconstitution of sterile dry powder forms of the contents within the media packets 20.

The applied material being tested can be any one or combination of the following: essential oils, hydrosols, tinctures, traditional Chinese herbs, Ayurvedic herbs, Silver ions, traditional or new antibiotic drugs or emulsion of nano-size essential oils in water.

The applied material can be provided in form of liquid or solid (powder) as long as it interacts with the sample.

Essential oil, includes ingredient(s), or mixtures from backhousia citriodora, cymbopogon citratus, cymbopogon martini, Eucalyptus globulus, lavender oil (such as lavandula angustifolia, lavandula latifolia, lavandula officinalis), mellisa officinalis, mentha piperita, melaleuca alternifolia, melaleuca cajuputi, ocimum basilicum, origamum vulgare, ocimum gratissimum, origanum vulgare ssp. Hirtum, oil of thyme (such as thymus capitatus, thymus vulgaris), oil of rosemary (such as Rosmarinus officinalis), syzygium aromaticum (clove), pelargonium graveolens (lemongrass), piper nigrum, pogostemon patchouli, santalum album (sandalwood), satureja montana, vetiveria zizanioides, pine-needle oil, aniseed oil, spruce-needle oil, eucalyptus oil, orange oil, lemon oil and emu oil (extracted from a bird), and other natural extracts listed in the following references: CRC Crit Rev Food Sci Nutr. 1977;9(4):345-73. Journal of Applied Microbiology, 1999 vol 86, No 6, pages 985-990 and essential oils as described in U.S. Pat. No. 6,280,751 issued Aug. 28, 2001 to Fletcher et al., herein incorporated by reference in its entirety.

Other essential oil compositions numerically listed herein include ingredient(s), or mixtures denoted herein in numeric sequence from: 1 Aniseed (Pimpinella anisum); 2 Angelica Root and Seed (Angelica archangelica); 3 Basil (Ocimum basilicum); 4 Bergamot (Citrus bergamia); 5 Black Pepper (Piper nigrum); 6 Cajeput (Melaleuca cajuputi); 7 Caraway (Carum carvi); 8 Cardamom (Elettaria cardamomum); 9 Cedarwood (Cedrus atlantica & C. deodara); 10 Chamomile, German (Chamomilla recutita); 11 Chamomile, Roman (Chamaemelum nobile); 12 Cinnamon Leaf (Cinnamomum zeylanicum); 13 Cistus (Cistus ladaniferus); 14 Citronella (Cymbopogon nardus); 15 Clary Sage (Salvia sclarea); 16 Clove Bud (Syzygium aromaticum); 17 Coriander Seed (Coriandrum sativum); 18 Cypress (Cupressus sempervirens); 19 Eucalyptus (Eucalyptus spp); 20 Everlasting (Helichrysum italicum); 21 Fennel, Sweet (Foeniculum vulgare); 22 Frankincense (Boswellia carteri); 23 Geranium (Pelargonium graveolens); 24 Ginger (Zingiber officinale); 25 Grapefruit (Citrus paradisi); 26 Ho (Cinnamomum camphora); 27 Hyssop (Hyssopus officinalis); 28 Jasmine (Jasminum officinalis); 29 Juniper Berry (Juniperus communis); 30 Laurel (Laurus nobilis); 31 Lavender, French (Lavandula stoechas); 32 Lavender, Spike (Lavandula latifolia); 33 Lavender, True (Lavandula angustifolia); 34 Lemon (Citrus limonum); 35 Lemongrass (Cymbopogon citratus); 36 Mandarin; Tangerine (Citrus reticulata); 37 Marjoram, Sweet (Origanum majorana); 38 Melissa (Melissa officinalis); 39 Myrrh (Commiphora myrrha); 40 Myrtle (Myrtus communis); 41 Neroli (Citrus aurantium ssp aurantium) (flos); 42 Niaouli (Melaleuca quinquenervia); 43 Orange (Citrus aurantium varieties); 44 Palmarosa (Cymbopogon martinii var motia); 45 Patchouli (Pogostemon cablin); 46 Peppermint (Mentha piperita); 47 Petitgrain (Citrus aurantium ssp aurantium) (fol); 48 Pine (Pinus spp); 49 Ravensara (Ravensara aromatica); 50 Rose (Rosa damascena); 51 Rosemary (Rosmarinus officinalis chemotypes); 52 Sage (Salvia spp); 53 Sandalwood (Santalum album); 54 Savory, Mountain (Satureia montana); 55 Spikenard (Nardostachys jatamansi); 56 Tea Tree (Melaleuca alternifolia); 57 Thyme (Thymus vulgaris chemotypes); 58 Vetiver (Vetiveria zizanioides); 59 Yarrow (Achillea millefolium); 60 Ylang Ylang (Cananga odorata); 61 Backhousia citriodora; 62 Cymbopogon citratus; 63 Cymbopogon martini; 64 Eucalyptus globulus; 65 Origamum vulgare; 66 Ocimum gratissimum; 67 Origanum vulgare ssp. Hirtum; 68 Thymus vulgaris; 69 Pogostemon patchouli; 70 Satureja Montana; 71 Vetiveria zizanioides; 72 Spruce-needle oil; and emu oil (extracted from a bird), and other natural extracts listed in the following references: CRC Crit Rev Food Sci Nutr. 1977;9(4):345-73. Journal of Applied Microbiology, 1999 vol 86, No 6, pages 985-990, herein incorporated by reference in its entirety.

In some cases it would be beneficial to exclude or reduce the concentration of some compounds such as ketones or phenols. Usually essential oils are only used up to 2% in volume, the rest of the oil is conducting or carrying oil that dissolve the essential oil(s). Example of conducting or carrying oils are Almond oil, Alovera gel, apricot kernel oil, avocado oil, borage seed oil, canola oil, carrot seed oil (cold-expressed),castor oil, cocoa butter, evening primrose oil, flaxseed oil, forah oil, glycerin, grape seed oil, hazelnut oil, jojoba oil, kuku nut oil, lecithin, macadamia nut oil, need oil, olive oil, oysternut oil, peanut oil, rapeseed oil, rose hip seed oil, safflower oil, sesame seed oil, shea butter, solubol, soybean oil, sunflower oil, and wheat germ oil. Like any other chemicals are beneficial in some concentration but harmful or inert in other. A method of removing selective compounds is disclosed in U.S. Pat. No. 7,250,185 given to Dowdle at el, herein incorporated by reference in its entirety. Other embodiments provide for the essential oils to contain stabilizer(s).

Hydrosol are water base extracts and may be manufactured as described in “Hydrosols: The Next Aromatherapy”, 2001, Catty, “Alcohol extract of Echinacea pallida reverses stress-delayed wound healing in mice” Zhai at el. Phytomedicine, 2009 Vol. 16, Issue 6, Pages 669-678, herein incorporated by reference in its entirety.

Tinctures or alcohol extracts may be made as described in “Antigonorrhoeal activity of plants used in Guatemala for the treatment of sexually transmitted diseases” Journal of Ethnopharmacology V 48, Issue 2, 1995, Pages 85-88, herein incorporated by reference in its entirety.

Silver ions may be made as described in Kreth at el. “The Antimicrobial Effect of Silver Ion Impregnation into Endodontic Sealer against Streptococcus mutans” Open Dent J. 2008; 2: 18-23 herein incorporated by reference in its entirety.

Traditional Chinese herbs: for example Rubricine was found to inhibit the growth of MDR including other microorganisms, as described in “Drug discovery and traditional Chinese medicine: science, regulation, and globalization” by Yuan 2001, ISBN 0792374118, 9780792374114 and Current Medicinal Chemistry, 2004, 11, 1423-1430 herein incorporated by reference in its entirety.

A list of plants and non plant materials denoted by the double asterisk (**) including their mixtures that can be used by category. The plant and non-plant categories are listed as within traditional treatment groups, for example “resolve the exterior”, “clear heat”, “resolve toxin”, “transform phlegm”, “invigorate blood” and other treatment groups shown below:

Warm, acrid herbs that resolve the exterior. The warm acrid herbs include Gui Zhi (Ramulus Cinnamomi Cassiae), Ma Huang (Herba Ephedrae), Fang Feng (Radix Ledebouriellae Divaricatae), Jing Jie (Herba seu Flos Schizonepetae Tenuifoliae), Qiang Huo (Rhizoma et Radix Notoptergygii), Zi Su Ye (Folium Perillae Frutescentis), Xi Xin (Herba cum Radice Asari), Bai Zhi (Radix Angelicae Dahuricae), and Sheng Jiang (Rhizoma Zingiberis Officinalis Recens).

Cool, acrid herbs that resolve the exterior. The cool, acrid herbs include Bo He (Herba Menthae Haplocalycis), Sheng Ma (Rhizoma Cimicifugae), Ju Hua (Flos Chrysanthemi Morifolii), Chai Hu (Radix Bupleuri), Ge Gen (Radix Puerariae), Sang Ye (Folium Mori Albae), Chan Tui (Periostracum Cicadae)**, and Niu Bang Zi (Fructus Arctii Lappae).

Clear Heat (qing re yao).

Drain Fire (xie huoyao) including Shi Gao (Gypsum)**, Zhi Mu (Rhizoma Anemarrhenae Asphodeloidis), Zhi Zi (Fructus Gardeniae Jasminoidis), Xia Ku Cao (Spica Prunellae Vulgaris), Dan Zhu Ye (Herba Lophatheri), Lu Gen (Rhizoma Phragmitis Communis), and Tian hua fen (Radix Trichosanthis Kirilowii).

Cool Blood (liang xue yao) including Sheng Di Huang (Radix Rehmanniae Glutinosae), Mu Dan Pi (Cortex Moutan Radicis), Chi Shao Yao (Radix Paeoniae Rubrae), Xuan Shen (Radix Scrophulariae Ningpoensis), Di Gu Pi (Cortex Lycii Radicis), Zi Cao (Radix Arnebiae seu Lithospermi), Shui Niu Jiao (Cornu Bubali)**, and Bai Wei (Radix Cynanchi Baiwei).

Clear Heat and Dry Dampness (qing re zao shi yao) including Huang Qin (Radix Scutellariae Baicalensis), Huang Bai (Cortex Phellodendri), Huang Lian (Rhizoma Coptidis),

### Long Dan Cao (Radix Gentianae Longdancao), and Ku Shen (Radix Sophorae Flavescentis).

Clear Heat and Resolve Toxin (qing re jie du yao) including Jin Yin Hua (Flos Lonicerae Japonicae), Lian Qiao (Fructus Forsythiae Suspensae), Pu Gong Ying (Herba Taraxaci Mongolici cum Radice), Bai Xian Pi (Cortex Dictamni Dasycarpi Radicis), Tu Fu Ling (Rhizoma Smilacis Glabrae), Ban Lan Gen (Radix Isatidis seu Baphicacanthi), Bai Hua She She Cao (Herba Hedyotidis Diffusae), Da Qing Ye (Folium Daqingye), and Zi Hua Di Ding (Herba cum Radice Violae Yedoensitis).

Clear Heat and Resolve Summerheat (qing re jie shu yao) Qing Hao (Herba Artemesiae Annuae), Bai Bian Dou (Semen Dolichoris Lablab), and Yin chai hu (Radix Stellariae Dichotomae).

Precipitants (xia yao).

Attacking Precipitants (gong xia yao) includes Da Huang (Radix et Rhizoma Rhei), and Mang Xiao (Mirabilitum)**.

Moist Precipitants (run xia yao) include Huo Ma Ren (Semen Cannabis Sativae), and Yu Li Ren (Semen Pruni).

Transform Dampness (hua shi yao) includes Cang Zhu (Rhizoma Atractylodis), Huo Xiang (Herba Agastaches seu Pogostemi), Sha Ren (Fructus Amomi), Hou Po (Cortex Magnoliae Officinalis), Bai Dou Kou (Fructus Amomi Kravanh), Cao Guo (Fructus Amomi Tsao-ko), and Pei Lan (Herba Eupatorii Fortunei).

Drain Dampness (li shi yao) includes Fu Ling (Sclerotium Poriae Cocos), Ze Xie (Rhizoma Alismatis Orientalis),Yi Yi Ren (Semen Coicis Lachryma-jobi), Che Qian Zi (Semen Plantaginis), Hua Shi (Talcum)**, Yin Chen Hao (Herba Artemesiae Yinchenhao), Bi Xie (Rhizoma Dioscoreae Hypoglaucae), Zhu Ling (Sclerotium Polypori Umbellati), Jin Qian Cao (Herba Lysimachiae), and Di Fu Zi (Fructus Kochiae Scopariae).

Dispel Wind and Eliminate Dampness (qu feng chu shi yao) includes Du Huo (Radix Angelicae Pubescentis), Qin Jiao (Radix Gentianae Qinjiao), Wei Ling Xian (Radix Clematidis), Cang Er Zi (Fructus Xanthii Sibirici), Hai Feng Teng (Caulis Piperis Futokadsurae), Sang Zhi (Ramulus Mori Albae), Sang Ji Sheng (Ramulus Sangjisheng), Xi Xian Cao (Herba Siegesbeckiae), and Wu Jia Pi (Cortex Acanthopanacis Gracilistyli Radicis).

Transform Phlegm, Suppress Cough and Calm Wheezing including to Dispel Cold and Transform Phlegm (qu han hua tan yao) including Ban Xia (Rhizoma Pinelliae Terenatae), Jie Geng (Radix Platycodi Grandiflori), Tian Nan Xing (Rhizoma Arisaematis), Xuan Fu Hua (Flos Inulae), and Bai Jie Zi (Semen Sinapis Albae).

Clear Heat and Transform Phlegm (qing re hua tan yao) include Qian Hu (Radix Peucedani), Zhe Bei Mu (Bulbus Fritillariae Thunbergii), Chuan Bei Mu (Bulbus Fritillariae Cirrhosae), Zhu Ru (Caulis Bambusae in Taeniis), Gua Lou (Fructus Trichosanthis),Gua Lou Ren (Semen Trichosanthis), and Kun Bu (Thallus Algae).

Suppress Cough and Calm Wheezing (zhi ke ping chuan yao) include Kuan Dong Hua (Flos Tussilaginis Farfarae), Bai Bu (Radix Stemonae), Su Zi (Fructus Perillae Frutescentis), Xing Ren (Semen Pruni Armeniacae), Sang Bai Pi (Cortex Mori Albae Radicis), Zi Wan (Radix Asteris Tatarici), and Pi Pa Ye (Folium Eriobotryae Japonicae).

Regulate Qi (Ii qi yao) include Chen Pi (Pericarpium Citri Reticulatae), Qing Pi (Pericarpium Citri Reticulatae Viride), Zhi Shi (Fructus Immaturus Citri Aurantii), Xiang Fu (Rhizoma Cyperi Rotundi), Chuan Lian Zi (Fructus Meliae Toosendan), Da Fu Pi (Pericarpium Arecae Catechu), and Wu Yao (Radix Lynderae Strychnifoliae).

Disperse Food and Guide Out Stagnation (xiao shi dao zhi yao) include Shen Qu (Massa Fermenta), Shan Zha (Fructus Crataegi), Lai Fu Zi Semen Raphani Sativi), Gu Ya (Fructus Oryzae Sativae Germinatus), Mai Ya (Fructus Hordei Vulgaris Germinatus), and Ji Nei Jin (Endothelium Comeum Gigerae Galli)**.

Invigorate Blood (huo xue yao) include Dan Shen (Radix Salviae Miltiorrhizae), Tao Ren (Semen Persicae), Hong Hua (Flos Carthami Tinctorii), Chuan Xiong (Radix Ligustici Chuanxiong), Chuan niu xi (Radix Achyranthis Bidentae), Huai Niu Xi (Radix Cyathulae Officinalis), Yu Jin (Tuber Curcumae), Yan Hu Suo Rhizoma Corydalis Yanhusuo), Ji Xue Teng (Radix et Caulis Jixueteng), Yi Mu Cao (Herba Leonuri Heterophylli), San Leng (Rhizoma Sparganii Stoloniferi), Mo Yao (Myrrha), Ru Xiang (Gummi Olibanum), E Zhu (Rhizoma Curcumae Ezhu), and Ze Lan (Herba Lycopi Lucidi).

Stop Bleeding (zhi xue yao) includes Ai Ye (Folium Artemisae Argyi), San Qi (Radix Notoginseng), Pu Huang (Pollen Typha), Di Yu (Radix Sanguisorbae Officinalis), Da Ji (Herba seu Radix Cirsii Japonici), Xiao Ji (Herba Cephalanoplos), Ou Jie (Nodus Nelumbinis Nuciferae Phizomatis), Ce Bai Ye (Cacumen Biotae Orientalis), Xian He Cao (Herba Agrimoniae Pilosae), and Bai Mao Gen (Rhizoma Imperatae Cylindricae).

Warm the Interior (wen li yao) includes Rou Gui (Cortex Cinnamomi Cassiae), Gan Jiang (Rhizoma Zingiberis Officinalis), Wu Zhu Yu (Fructus Evodiae Rutaecarpae), and Ding Xiang (Flos Caryophylli).

Tonify Qi (bu qi yao) include Ren Shen (Radix Ginseng), Dang Shen (Radix Codonopsitis Pilosulae), Bai Zhu (Rhizoma Atractylodis Macrocephalae), Huang Qi (Radix Astralagi Membranaceus), Shan Yao (Radix Dioscoreae Oppositae), Da Zao (Fructus Zizyphi Jujubae), Tai Zi Shen (Radix Pseudostellariae Heterophyllae), and Gan Cao (Radix Glycyrrhizae Uralensis).

Tonify Yang (bu yang yao) include Xu Duan (Radix Dipsaci Asperi), Du Zhong (Cortex Eucommiae Ulmoidis), Bu Gu Zhi (Fructus Psoraleae Corylifoliae), Tu Si Zi (Semen Cuscutae Chinensis), Rou Cong Rong (Herba Cistanches Deserticolae), Lu Rong (Cornu Cervi Parvum)**, Yi Zhi Ren (Fructus Alpiniae Oxyphyllae), Ba Ji Tian (Radix Morindae Officinalis),Yin Yang Huo (Herba Epimedii), Dong Chong Xia Cao (Cordyceps Sinensis), and Xian Mao (Rhizoma Curculiginis Orchioidis).

Tonify Blood (bu xue yao) include Dang Gui (Radix Angelicae Sinensis), Bai Shao Yao (Radix Paeoniae Lactiflorae), He Shou Wu (Radix Polygoni Multiflori), Shu Di Huang (Radix Rehmanniae Glutinosae Conquitae), Long Yan Rou (Arilllus Euphoriae Longanae) and E Jiao (Gelatinum Corii Asini)**.

Tonify Yin (bu yin yao) include Mai Men Dong (Tuber Ophipogonis Japonici), Tian Men Dong (Tuber Asparagi Cochinchinensis), Sha Shen (Radix Adenophorae seu Glehniae), Nu Zhen Zi (Fructus Ligustri Lucidi), Bai He (Bulbus Lilii), Gou Qi Zi (Fructus Lycii),Gui Ban (Plastrum Testudinis)**, Bie Jia (Carapax Amydae Sinensis)**, Yu Zhu (Rhizoma Polugonati Odorati), Han Lian Cao (Herba Ecliptae Prostratae), Hei Zhi Ma (Semen Sesami Indici), and Huang Jing (Rhizoma Polygonati).

Stabilise and Bind (gu se yao) includes Wu Wei Zi (Fructus Schisandrae Chinensis), Shan Zhu Yu (Fructus Comi Officinalis), Lian Zi (Semen Melumbinis Nuciferae), Fu Pen Zi (Fructus Rubi Chingii), Ma Huang Gen (Radix Ephedrae), Qian Shi (Semen Euryales Ferocis), Fu Xiao Mai (Semen Tritici Aestivi), Rou Dou Kou (Semen Myristicae Fragrantis), and Wu Mei (Fructus Pruni Mume).

Calm the Liver and Extinguish Wind (ping gan xi feng yao) include Gou Teng (Ramulus cum Uncis Uncariae), Bai Ji Li (Fructus Tribuli Terrestris), Shi Jue Ming (Concha Haliotidis)**, Jiang Can (Bombyx Batrycatus)**, and Di Long (Lumbricus)**.

Calm the Spirit including that which Nourishes the Heart and Calms the Spirit (yang xin an shen yao) includes Yuan Zhi (Radix Polygalae Tenuifoliae), Suan Zao Ren (Semen Zizyphi Spinosae), Bai Zi Ren (Semen Biotae Orientalis), He Huan Pi (Cortex Albizziae Julibrissin), and Ye Jiao Teng (Caulis Polygoni Multiflori).

Settle the Spirit (zhen an yao) includes Long gu (Os Draconis)**, Mu li (Concha Ostreae)**, Ci shi (Magnetitum)**, and Zhen zhu mu (Concha Margaritaferae)**.

Open the Orifices (kai qiao yao) includes, Shi Chang Pu (Rhizoma Acori Graminei), Bing Pian (Bomeol) and An Xi Xiang (Benzoinum).

Resolve the Exterior (jie biao ji) include Ma Huang Tang—Ephedra Decoction,Gui Zhi Tang—Cinnamon Twig Decoction, Yin Qiao San—Honeysuckle & Forsythia Powder, Sang Ju Yin—Mulberry Leaf & Chrysanthemum Decoction, Xiao Qing Long Tang—Minor Bluegreen Dragon Decoction, Ren Shen Bai Du San—Ginseng Powder to Overcome Pathogenic Influences, Ge Gen Tang—Kudzu Decoction, Cang Er Zi San—Xanthium Powder, and Chai Ge Jie Ji Tang—Bupleurum and Kudzu Decoction to Release the Muscle Layer.

Clear Heat (qing re ji) includes Huang Lian Jie Du Tang—Coptis Decoction to Relieve Toxicity Xie Bai San—Drain the White Powder, and Shao Yao Tang—Peony Decoction**.

Drain Downward (xie fa ji) includes Xiao Cheng Qi Tang—Minor Order the Qi Decoction, and Ma Zi Ren Wan—Hemp Seed Pill.

Harmonise (he ji) includes Xiao Chai Hu Tang—Minor Bupleurum Decoction, Xiao Yao San—Rambling Powder, Si Ni San—Frigid Extremities Powder, and Ban Xia Xie Xin Tang—Pinellia Decoction to Drain the Epigastrium.

Expel Dampness (qu shi ji) includes Wu Ling San—Five-Ingredient Powder with Poria, Zhu Ling Tang—Polyporus Decoction, Wu Pi San—Five Peels Powder, Ping Wei San—Calm the Stomach Powder, Huo Xiang Zhen Qi San—Agastache Powder to Rectify the Qi, Ba Zheng San—Eight-Herb Powder for Rectification, and Er Miao San—Two-Marvel Powder.

Warm the Interior (wen li ji) includes Li Zhong Wan—Regulate the Middle Pill, Wu Zhu Yu Tang—Evodia Decoction, Da Jian Zhong Tang—Major Construct the Middle Decoction, and Xiao Jian Zhong Tang—Minor Construct the Middle Decoction.

Tonify (bu ji) includes Si Jun Zi Tang—Four-Gentlemen Decoction, Liu/Xiang Sha/Liu Jun Zi Tang—Six Gentlemen Decoction et al., Bu Zhong Yi Qi Tang—Tonify the Middle a & Augment Qi Decoction, Ba Zhen Tang/Yi Mu Ba ZhenTang—Eight-Treasure Decoction et al., Shi Quan Da Bu Tang—All-Inclusive Great Tonifying Decoction, Liu Wei Di Huang Tang—Six-Ingredient Deocotion with Rehmannia, (Zhi Bai Di Huang Tang/Qi Ju Di Huang Tang/Du Qi Wan/Mai Wei Di Huang Tang), Zuo Gui Wan—Restore the Left (Kidney) Pill, Er Xian Tang—Two-Immortal Decoction, Si Wu Tang—Four-Substance Decoction, (Tao Hong Si Wu Tang/Qin Lian Si Wu Tang), Zhi Gan Cao Tang—Honey-Fried LicoriceDecoction, Gui Pi Tang—Restore the Spleen Decoction, Dang Gui Shao Yao San—Tangkuei & Peony Powder, Shao Yao Gan Cao Tang—Peony & Licorice Decoction, Shen Ling Bai Zhu San—Ginseng, Poria,& Atractylodes Macrocephala Powder, Ren Shen Yang Rong Wan—Ginseng Decoction to Nourish the Nutritive Qi, Dang Gui Bu Xue Tang—Dang Gui Decoction to Tonify the Blood, Sheng Mai San—Generate the Pulse Powder, Yi Wei Tang—Benefit the Stomach Decoction, and Yi Guan Jian—Linking Decoction.

Transform Phlegm (hua tan ji) includes Er Chen Tang—Two-Cured Decoction, Wen Dan Tang—Warm the Gallbladder Decoction, Zhi Sou San—Stop Coughing Powder, Ban Xia Bai Zhu Tian Ma Tang—Pinellia, Atractylodes Macrocephalea, and Gastrodia Decoction**, and Bei Mu Gua Lou San—Fritillaria and Trichosanthes Fruit Powder.

Regulate Qi (li qi ji) includes Ban Xia Hou Po Tang—Pinellia and Magnolia Bark Decoction, Yue Ju Wan—Escape Restraint Pill, Su Zi Jiang Qi Tang—Perilla Fruit Decoction for Directing Qi Downward, Ding Chuan Tang—Arrest Wheezing Decoction, and Ju Pi Zhu Ru Tang—Tangerine Peel and Bamboo Shaving Decoction.

Invigorate Blood (huo xue ji) includes Xue Fu Zhu Yu Tang—Drive Out Stasis in the Mansion of Blood Decoction (and variants), Gui Zhi Fu Ling Wan—Cinnamon Twig and Poria Pill, Wen Jing Tang—Warm the Menses Decoction, Dan Shen Yin—Salvia Decoction, and Tao He Cheng Qi Tang—Peach Pit Decoction to Order the Qi.

Calm the Spirit (an shen ji) includes Suan Zao Ren Tang—Sour Jujube Decoction, and Gan Mai Da Zao Tang—Licorice, Wheat, Jujube Decoction.

Extinguish Wind (xi feng ji) includes Tian Ma Gou Teng Yin—Gastrodia & Uncaria Decoction**, Du Huo Ji Sheng Tang—Angelica Pubescens and Sangjisheng Decoction, Juan Bi Tang—Remove Painful Obstruction Decoction, and Di Huang Yin Zi—Rehmannia Decoction.

Disperse Food and Guide Out Stagnation (xiao shi dao zhi ji) includes Bao He Wan—Preserve Harmony Pill.

Stabilise and Bind (gu se ji) includes Yu Ping Feng San—Jade Windscreen Powder, Si Shen Wan—Four-Miracle Pill, and Suo Quan Wan—Shut the Sluice Pill.

Moisten Dryness (run zao ji) includes Xing Su San—Apricot Kernel and Perilla Leaf Powder, and Mai Men Dong Tang—Ophiopogonis Decoction.

Open the Orifices (kai qiao ji) includes Di Tan Tang—Scour Phlegm Decoction.

Expel Parasites (qu chong ji) includes Wu Mei Wan—Mume Pill. Ayurvedic herbs: Numerically listed herein are ayurvedic herbs that are denoted by their Sanskrit and botanical names: 1 Adhah puspī Trichodesma indicum, (Linn) R. Br.; 2 Adhaki Cajanus cajan, (Linn) Mills; 3 Agaru Aquilaria agallocha, Roxb; 4 Agastya Sesbenia grandiflora, Pers.5 Agni manthā Premna integrifolia, Linn; 6 Ahi phēna Papver somniferum, Linn; 7 Ajmodā Apium graveolens, Linn; 8 Ākārkarabha Anacyclus pyrethrum, DC; 9 Ākhuparnī Ipomoea reniformis, Chois; 10 Aksotaka Juglans regia, Linn.; 11 Āmalakī Emblica officinalis, Gaertn.; 12 Amarvallī Cuscuta reflexa, Roxb.; 13 Amlavetasa Garcinia pedunculata, Roxb.; 14 Amlikā Tamarindus indica, Linn.; 15 Amra Mangifera indica, Linn.; 16 Āmra haridra Curcuma amada, Roxb; 17 Ankota Alangium Salvifolium, (Lin. f.) Wang.; 18 Apāmārga Achyranthes aspera, Linn.; 19 Aparājitā Clitoria temeata, Linn.; 20 Āragvadha Cassia fistula, Linn.; 21 Aralu Ailanthus excelsa, Roxb.; 22 Ara [symbol] ya jīraka Centrotherum anthelminticum, (Linn) Kurtze.; 23 Ārdraka Zingiber officinale, Roscoe.; 24 ArisŃaka Sapindus trifoliatus, Linn.; 25 Arjuna Terminalia arjuna, (Roxb) Wight & Arn.; 26 Arka Calotropis giganticea, R. Br.; 27 Aśmantaka Ficus rumphii, Blume.; 28 Aśoka Saraca asoca (Roxb) De Wilde.; 29 AsŃisamhāraka Vitis quadrangularis, Linn.; 30 Aśvagandha Withania somnifera, Dunal.; 31 Aśvagola Plantago ovata, Forsk.; 32 Aśvakar [symbol] a Dipertocarpus turbinatus, Gaertn. f.; 33 Aśvattha Ficus religiosa, Linn.; 34 Atasī Linum usitaissimum, Linn.; 35 Ati balā Abutilon indicum, (Linn.) Sw.; 36 Ativisā Aconitum heterophyllum, Wall. ex Royle.; 37 Āvartakī Cassia auriculata, Linn.; 38 Āvartanī Helicteres isora, Linn.; 39 Ayapānam Eupatorium triplinerve, Vahl.; 40 Babbūila Acacia arabica, Wild.; 41 Badara Zizyphus mauritiana, Lamk.; 42 Bākucī Psoralea corylifolia, Linn.; 43 Bakula Mimusops elingi, Linn.; 44 Balā Sida cordifolia, Linn.; 45 Bañigā Canabis sativa, Linn.; 46 Bhallātaka Semecarpus anacardium, Linn.; 47 Bha [symbol] dīra Clerodendrum infortunatum, Linn.; 48 Bhārńgī Clerondendron serratum, Spreng.; 49 Bh [symbol] ńgarāja Eclipta erecta, Linn.; 50 Bhūmyāmlakī Phyllanthus niruri, Sensu Hook. f.; 51 Bhūrja Betula utilis, D. Don.; 52 Bīja pūraka Citrus medica, Linn.; 53 Bījaka Pterocarpus marsupium, Roxb.; 54 Bilva Aegle marmelos, Corr.; 55 Bimbī Coccinia grandis, (Linn) Voigt.; 56 Bola Commiphora myrrh, Nees.; 57 Brāhmī Bacopa moniera, (Linn.) Pennell.; 58 B [symbol] hatī Solanum indicum, Linn.; 59 Cakramarda Cassia tora, Linn.; 60 Caksusya Cassia absus, Linn.; 61 Campaka Michelia champak, Linn.; 62 Cañcu Corchorus fascicularis, Lam.; 63 Candana Santalum alba, Linn.; 64 Cańidraśūra Lepidium sativum, Linn.; 65 Cāñgērī Oxalis corniculata, Linn.; 66 Cauhāra Artimisia maritime, Linn.; 67 Cavya Piper chaba, Hunter.; 68 Chatraka Agaricus campestras, Linn.; 69 Chikkikā Centipeda minima, (L.) Willd.; 70 Chukra Rumex vesicarius, Linn.; 71 Ciribilva Holoptelea integrifolia, (Roxb) Planch.; 72 Citraka mūla Plumbago zeylanica, Linn.; 73 Coraka Angelica glauca, Edgw.; 74 Dādima Punica granatum, Linn.; 75 Damanaka Artemisia vulgaris, Linn.; 76 Danti mūla Baliospermun montanum, Muell-Arg.; 77 Dāru haridrā Berberis aristata, DC.; 78 Devadāru Cedrus deodara, Roxb.-Loud; 79 Dhāmārgava Luffa aegyptica, Mill ex Hook. f.; 80 Dhanvana Grewia tiliaefolia, Vahl.; 81 Dhānyaka Coriandrum sativum, Linn.; 82 Dhātakī Woodfordia floribunda, Salisb.; 83 Dhattūra Dhattura metel, Linn.; 84 Dhava Anogeissus latifolia, (Roxb. ex DC) Bedd.; 85 Drāksa Vitis vinifera, Linn.; 86 Dro [symbol] a puspī Leucas cephalotus, Spreng.; 87 Dugdhaphenī Taraxacum officinale, Weber.; 88 Duralabha Fagonia cretica, Linn.; 89 Dūrvā Cynodon dactylon, Pers.; 90 Dvīpāntara vacā Smilax glabra, Roxb.; 91 Ēla Elettaria cardamomum, Maton.; 92 Ēra [symbol] da Ricinus communis, Linn.; 93 Era [symbol] da karkatī Carica papaya, Linn.; 94 Gambhārī Gmelina arboria, Linn.; 95 Gandha prasāranī Paederia foetida, Linn.; 96 Gāngerukī Grewia populifolia, Vahl.; 97 Giri parpaŃī Podophyllum hexandrum, Royle.; 98 Gojivhā Onosma bracteatum, Wall.; 99 Gōksura Tribulus terrestris, Linn.; 100 Goraksa Dlabergia lanceolaria, Linn.; 101 Goraksamajjā Aerva lanta, (Linn) Juss.; 102 Guda śarkarā Grewia hirsute, Vahl.; 103 Gudūcī Tinospora cordifolia, Willd Meirs.; 104 Guggulu Commiphora mukul, Hook.; 105 Guñajā Abrus precatorius, Linn.; 106 Hamsarāja Adiantum lunulatum, Burm. f.; 107 Hapusā Juniperus communis, Linn.; 108 Haramala Paganum harmal, Linn.; 109 Haridrā Curcuma longa, Linn.; 110 Haridru Adina cordifolia, Hook. f.; 111 Harītakī Terminalia chebula, Retz.; 112 Hijjala Baringtonia acutangula, Gaertn.; 113 Hińigu Ferula asafoetida, Linn.; 114 Hinsrā Capparis sepiaria, Linn.; 115 H [symbol] tpatrī Digitalis purpurea, Linn.; 116 Iksvāku Lagenaria siceraria, Standle.; 117 Indravārunī Citrulus colocynthis, Schard.; 118 Ińgudī Balanites aegyptica, (Linn.) Dell.; 119 Irimeda Acacia farnesiana, Willd.; 120 Jambū Eugenia jambolana, Lam.; 121 JaŃāmansī Nordostachys jatamansi, Dc.; 122 Jātī Jasminum grandiflorum, Linn.; 123 Jātīphala Myristica fragrans, Houtt.; 124 Jayanti Sesbania aegyptica, Pers; 125 Jayapāla Croton tiglium, Linn.; 126 Jhe [symbol] dū Tagetus erecta, Linn.; 127 Jīmutaka Luffa echinata, Roxb.; 128 Jīraka Cuminum cyminum, Linn.; 129 Jīvantī Leptadenia reticulate, (Retz.) Wight & Arn.; 130 Jūphā Hyssopus officinalis, Linn.; 131 Jyōtismatī Celastrus paniculata, Willd.; 132 Kadali Musa paradisiacal, Linn.; 133 Kadamba Anthocephalus indicus, A. Rich.; 134 Kākamacī Solanum nigrum, Linn.; 135 Kākodumbara Ficus hispida, Linn.; 136 Kālamegha Andrographis paniculata, (Burm. f.) Wall.; 137 Kamala Nelumbo nucifers, Gaertn.; 138 Kampillaka Mallotus philippinensis, Muell Arg.; 139 Kāñchanāra Bauhinia variegate, Linn.; 140 Kāndīra Ranunculus sceleratus, Linn.; 141 Kańkōla Piper cubeba, Linn.; 142 Kankushtha Garcinia morella, Desr.; 143 Ka [symbol] Ńakādī Solanum xanthocarpum, Sachrd & Wendl.; 144 Kapikacchu Mucuna prurita, Hook.; 145 Karamarda Carissa congesta, Linn.; 146 Karañja Pongamia pinnata, (Linn.) Merr.; 147 Kāravellaka Momordica charantia, Linn.; 148 Karavīra Nerium indicum, Soland.; 149 Karīra Capparis deciduas, Edgew.; 150 Kar [symbol] aŃaśkńgī Pistacia intererima, Stew. ex. Brandis.; 151 Karmaranga Averrhoa carambola, Linn.; 152 Kārpāsa Gossypium herbaceum, Linn.; 153 Karpūra Cinnamomum camphora, Nees & Eberm.; 154 Kāśa Saccharum spontaneum, Linn.; 155 Kāsamarda Cassia occidentalis, Linn.; 156 Kāsani Chicorium intybus, Linn.; 157 Kaśeruka Scirpus grosus, Linn.; 158 KāsŃa dāru Polyalthia longifolia, Benth & Hook.f.; 159 Kataka Strychnos potatorum, Linn. f.; 160 KaŃphala Myrica nagi, Thunb.; 161 Katu vīrā Capsicum annum, Linn.; 162 KaŃukī Picrorrhiza Kurroa, Royle ex Benth.; 163 KaŃutumbī Lagenaria leucantha, (Duch.) Rusby.; 164 Kebuka Costus speciosus, Koen. ex Retz.; 165 Ketakī Pandanus tectorius, Soland ex Parkino.; 166 Khadira Acasia catechu, Willd.; 167 Kharjūra Phoenix sylvestris, Roxb.; 168 Khatmī Althaea officinalis, Linn.; 169 Khūbakalā Sisymbrium irio, Linn.; 170 Kirātatikta Swertia chirata, Buch-Ham.; 171 KīŃamārī Aristalochia bracteolate, Lamk.; 172 Kokilāksa Astercantha longifolia, Nees.; 173 Kośāmra Schleichera trijuga, Willd.; 174 Kośātakī Luffa acutangulata, (Linn.) Roxb.; 175 K [symbol] s [symbol] a bīja Ipomoea hederacea, Linn-Jacq.; 176 K [symbol] s [symbol] a jīraka Carum carvi, Linn.; 177 Kukundara Blumea lacera, D. C.; 178 Kulattha Dolichos biflorus, Linn.; 179 Kumādī Aloe vera, Linn.; 180 Kumbhīka Careya arborea, Roxb.; 181 Kumuda Nymphea alba, Linn.; 182 Kuńkuma Crocus sativus, Linn.; 183 Kupīlu Strychnos nux-vomica, Linn.; 184 Kuśa Desmostachya bipinnata, Stap. f.; 185 Kūsmā [symbol] da Benincasa hispida, Thum.; 186 KusŃha Saussurea lappa, C. B. Clarke.; 187 KuŃaja Holarrhena antidysentrica, Wall. ex. D C.; 188 Lajjālu Mimosa pudica, Linn.; 189 Lāńgalī Glosiosa superba, Linn.; 190 Latā karañja Caesalpinia bonduc, Roxb.; 191 Latā kastūrī Hibiscus abelmoschus, Linn.; 192 Lavańga Syzygium aromaticum, Merr perry.; 193 Lodhra Symplocos racemosa, Roxb.; 194 Madana phala Randia dumetorum, Lam.; 195 Madayantikā Lawsonia inermis, Linn.; 196 Madhūkā Madhuka indica, Jf. Gmel.; 197 MadhuyasŃi Glycerrhiza glabra, Linn.; 198 Mahābharī vacā Alpinia galangal, Willd.; 199 Makhāna Euryale ferox, Salisb.; 200 Mamīra Thalictrum foliolosum, Dc.; 201 Māmsarohi [symbol] i Soymida febrifuga, A. Juss.; 202 Māna kanda Alocasia indica, Scholt.; 203 Ma [symbol] dūka par [symbol] ī Centella asiatica, (Linn.) Urban.; 204 MañjisŃa Rubia cordifolia, Linn.; 205 Marica Piper nigrum, Linn.; 206 Mārka [symbol] dikā Cassia angustifolia, Vahl.; 207 Masa par [symbol] ī Teramus labialis, Spreng; 208 Māyāphala Quercus infectoria, Roxb.; 209 Mayūrśikhā Adiantum caudatum, Linn.; 210 Medaśāka Litsea chinensis, Lam.; 211 Mesa ś [symbol] ngī Gymnema sylvestre, R. Br.; 212 Methikā Trigonella foenum-graecum, Linn.; 213 Miśreyā Foeniculum vulgare, Mill.; 214 Mucakunda Pterospermum diversifolium, Blume.; 215 Mudga par [symbol] ī Phaseolous trilobus, Ait.; 216 Mūlaka Raphanus sativus, Linn.; 217 Mu [symbol] dī Sphaeranthus indicus, Linn.; 218 Mūrvā Marsdenia tenacissima, Wight & Arn.; 219 Muśalī Asparagus adscendens, Roxb.; 220 Mustaka Cyperus rotundus, Linn.; 221 Nādī hiñgu Gardenia gummifera, Linn.F.; 222 Nāga dantī Croton oblongifolus, Roxb.; 223 Nāgabalā Sida veronicaefolia, Lam.; 224 Nāgadamanī Crinum asiaticum, Roxb.; 225 Nāgakesar Mesua ferrea, Benth-Hook.; 226 Nala Arundo donax, Linn.; 227 Nīlī Indigofera tinctoria, Linn.; 228 Nimba Azadirachta indica, Linn.; 229 Nimbuka Citrus limetta, Wight & Arn; 230 Nirgu [symbol] dī Vites negundo, Linn.; 231 Nirvisā Delphinium denudatum, Wall. ex. Hook. f.; 232 Padmaka Prunus cirasoidus, D. Don.; 233 Palāndu Allium cepa, Linn.; 234 Palāśa Butea monosperma, Lam-Kutze.; 235 Panasa Artocarpus integrifolia, Linn. f.; 236 Pārasīka yavānī Hyocyamus niger, Linn.; 237 Pāribhadrā Erithrina variegate, Linn.; 238 Pārijāta Nyctanthus arbortristis, Linn.; 239 Pārīsa Thespesia populnea, (Linn) Soland ex Corr.; 240 Par [symbol] a bīja Kalanchoe pinneta, Pers.; 241 Par [symbol] a yavānī Coleus aromaticus, Benth.; 242 Parpata Fumaria parviflora, Lamk.; 243 Parūsaka Grewia asiatica, Linn.; 244 Pāsā [symbol] abheda Bergenia lingulata, (Wall.) Engl.; 245 PāŃalā Stereospermum suaveolens, Dc.; 246 Pātāla garudī Cocculus hirsutus, (Linn.) Diels; 247 Pāthā Cissempelus pareira, Linn.; 248 PaŃōla Trichosanthes dioica, Roxb.; 250 Pīlu Salvadora persica, Linn.; 251 Pippali Piper longum, Linn.; 252 Piśāca kārpāsa Abroma augusta, Linn. f.; 253 Pīta mūlā Rueum emodi, Wall.; 254 Plaksa Ficus lacor, Buch-Ham.; 255 Priyāla Buchanania latifolia, Roxb.; 256 Priyańigu Callicarpa macrophylla, Vahl.; 257 P [symbol] sni par [symbol] ī Ureria picta, Desv.; 258 Pūga Areca catechu, Linn.; 259 Punamavā Boerhavia diffusa, Linn.; 260 Punnāga Calophyllum inophyllum, Linn.; 261 Puskara mūla Inula racemosa, Hook. f.; 262 Pūtīhā Mentha piperata, Linn.; 263 Putrañjīevaka Putranjiva roxburghii, Wall.; 264 Rājikā Brassica juncea, Linn.; 265 Rakta chanda Pterocarpus santalinus, Linn. f.; 266 Rakta [symbol] iryāsa Daemenorops draco, Blume.; 267 Rāsnā Alpinia officinarum, Hance.; 268 Rasōna Allium sativum, Linn.; 269 Rohisa Cymbopogon schoenanthus, Linn.; 270 Rohītaka Tecomella undulate, G. Don.; 271 Rumī mastagī Pistacia lentiscus, Linn.; 272 Sahadēvī Vernonia cineria, Len.; 273 Sailēya Parmelia perlata, Ach.; 274 Saireyaka Barleria prionitis, Linn.; 275 Śaivāla Ceratophyllum demersum, Linn.; 276 Śāla Shorea robusta, Gaertn.; 277 Śālapar [symbol] ī Desmodium gangeticum, D. C.; 278 Śallakī Boswellia serrata, Roxb.; 279 Śālmalī Bombax malabaricum, Schott and Endl.; 280 Śamī Prosopis cineraria, (Linn.) Druce.; 281 Śa [symbol] a puspī Crotalaria verrucosa, Linn.; 282 Śańka puspī Convolvulus pluricaulis, Chois.; 283 Sapta cakra Salacia reticulate, Wight.; 284 Saptapar [symbol] a Alstonia scholaris, R. Br.; 285 Śara Sacchrum munja, Roxb.; 286 Sarala Pinus-cedrus deodar, Roxb. Sargent; 287 Śarapuńkhā Tephrosia purpurea, Linn.; 288 Sārivā Hemidesmus indicus, R. Br.; 289 Sarja Vateria indica, Linn.; 290 Sarpagandhā Rauwolfia serpentine, Benth ex Kurza; 291 Sarsapa Brassica campestris, Linn.; 292 Śata puspā Anethum sowa, Kurtz.; 293 Śatāvarī Asparagus racemosus, Willd.; 294 ŚaŃhī Hedychium spicatum, Hamilt ex Smith.; 295 Satyānasī Argemone mexicana, Linn.; 296 Śigru Moringa oleifera, Lam.; 297 Silhaka Altingia excelsa, Noronha.; 298 Śińśipā Dalbergia sissoo, Roxb.; 299 Śirīsa Albizzia lebbek, Benth.; 300 Sītāphala Anona squamosa, Linn.; 301 Ślesmātaka Cordia dichotoma, Forst. f.; 302 Snuhī Euphorbia nerifolia, Linn.; 303 Soma Ephedra gerardiana, Wall.; 304 Ś [symbol] ńgātaka Trapa bispinosa, Roxb.; 305 Sudarśana Crinum latifolium, Linn.; 306 Suddāma Ruta graveolens, Linn; 307 Sugandha vāstūka Chenopodium ambrosioides, Bert. ex. Steud.; 308 Sura punnāga Ochrocarpus longifolia, Benth & Hook. f.; 309 Sūra [symbol] a Amorphophallus campanulatus, Roxb.; 310 Surañjāna Colchicum luteum, Baker.; 311 Svar [symbol] aksīfī Euphorbia thomsoniana, Boiss.; 312 Śyōnāka Oroxylum indicum, Vent.; 313 Tagara Valeriana wallichi, D C.; 314 Taila par [symbol] ī Eucalyptus globules, Labill.; 315 Tāla Borassus flabellifera, Linn.; 316 Tālamūlī Curculigo orchioides, Gaertn.; 317 Talispatrī Abies webbiana, Lindle.; 318 Tāmrapar [symbol] a Nicotiana tobacum, Linn.; 319 Taru [symbol] ī Rosa centifolia, Linn.; 320 Tavaksīfī Curcuma angustifolia, Roxb.; 321 Tējovhā Zantho-xylon alatum, Roxb.; 322 Tejpatra Cinnamomum tamala, Nees.; 323 Tila Sesamum indicum, D C.; 324 Tila parni Cleome gyanadra, Briquet.; 325 Tinduka Diospyros tomentosa, Roxb.; 326 Tiniśa Ougeinia dalbergioides, Benth.; 327 Tintidika Rhus parviflora, Roxb.; 328 Tōdarī Lepidium iberis, Linn.; 329 Trapusa Cucumis sativus, Linn.; 330 Trāyamāna Gentiana Kurroa, Royle.; 331 Triv [symbol] t Operculina ipomoea, Linn.; 332 Tulasī Ocimum sanctum, Linn.; 333 Tūta Morus alba, Linn.; 334 Tuvaraka Hydnocarpus wightana, Blume.; 335 Twak Cinamonum zeylonica, Blume.; 336 Udumbara Ficus racemosa, Linn.; 337 Unnāva Zizyphus

sativa, Gaertn.; 338 Upakun\check{c}ikā Nigella sativa, Linn.; 339 Uśavā Similax zeylanica, Linn.;

340 Uśīra Vetiveria zizanioides, L. Nash.; 341 UŃańga [symbol] a Blepharis edulis, Pers.; 342 Vacā

Acorus calamus, Linn.; 343 Vamśa Bambusa arundinaceae, Willd.; 344 Vana palāndu Urginea indica, Kunth.; 345 Vanapsa Viola odorata, Linn.; 346 Vandāka Dendrophthoe falcate, Linn.; 347 Vārāhī kanda Dioscoria sativa, Linn.; 348 Varu [symbol] a Crataeva nurvala, Roxb.; 349 Vāsā Adhatoda vasica, Nees.; 350 Vata Ficus bengalensis, Linn.; 351 Vātāda Prunus amygdalus, Beill.; 352 Vatsanābhī Aconitum ferox, Wall.; 353 Vibhītakī Terminalia belerica, Roxb.; 354 Vidańga Embelia ribes, Burm. f.; 355 Vidādī kanda Pueraria tuberose,

D C.; 356 Vīkañkata Flacourtia ramontchi, L. Herit.; 357 Vīrataru Dichrostachys cinerea,

(Linn) Wight & Arn.; 358 V [symbol] ddhādāru Argyria speciosa, Sweet.; 359 Vrukshāmla Garcinia

indica, Chois.; 360 Vyaghranakhī Capparis zeylanica, Linn.; 361 Yavānī Trachispermum

amami, Linn. Sprague; 362 Yavāsa Alhaji mourorum, (Bieb) Desv; and 363 Yuthicā Jasminum auriculatum, Vahl.

Nanometer size particle dissolved in water. The benefit of a nanometer formulation is the ability of a hydrophobic material to penetrate a the epidermis layer of the skin. The method of preparation is disclosed in U.S. Pat. No. 5,091,187, given to Haynes.

In alternative embodiments the panel of applied material Dropper bottles 32 may be replaced by applied material disks. The applied material disks may be either prepared just prior to application to inoculated plates by application of essential oils applied with at least one drop, or other sufficient volume, from the essential oil bottles 32, or may be obtained preloaded with sufficient quantities of applied material. In general sufficient quantities of applied materials denotes that amount of applied material having a sufficient mass-concentration activity to effect the production of inhibition zones to bacterial or fungal standard cultures similar to those obtainable from the ATCC or other strain repositories that are representative of what may be cultured from human inoculums. The applied material disks 232 may also be in the form of strips to conduct gradient responses. The applied material disks 32 and strips then may be placed directly on the inoculated agars plates 14. Zone inhibition measurements then can be made as described by U.S. Pat. No. 4,701,850 to Gibbs, herein incorporated by reference in its entirety.

In case where disk or strip are used they need to be secured to the surface or the Petri dish needs to be placed facing up, in this case covered with a lead.

In addition the applied material can include stabilizers to ensure longer shelf life. Stabilizers are disclosed in U.S. Pat. No. 2,282,808 given to Musher, herein incorporated by reference in its entirety.

Image analysis algorithm generally consists transferring the color image into gray scale, then applying second a Floyd-Steinberg dithering step, and enumerating the connected black pixels as representing the area of the inhibition zone. The area is a linear function of the inhibition capability of the medicine. Floyd-Steinberg step is disclosed in R. W. Floyd, L. Steinberg, An adaptive algorithm for spatial grey scale. Proceedings of the Society of Information Display 17, 75-77 (1976), herein incorporated by reference in its entirety.

An alternate embodiment of the image analysis algorithm includes converting the color image to gray scale followed by applying Floyd-Steinberg dithering. Clusters of black pixels are created by joining or merging neighboring black pixels that occupy the inhibition zone. are joined neighboring pixels are enumerated. For each pixel cluster is assigned a cluster number. If the distances between two clusters is 1 the pixels are assigned a lower cluster number. Thereafter the process is repeated until there are no more merging to be done of the neighboring black pixels. The clusters are re-evaluated and re-assigned cluster numbers so that the first is 1 and the last is N. The center of each cluster is calculated by letting the sum (x,y) be (0,0). For each intensity I=sum+(Xi, Yi) and the center=sum/n. As there are N points in each cluster, each pixel has a different location (Xi,Yi), so that the sum a two dimensional vector. The cluster size and cluster center is reported and onto the image the cluster size and the cluster central poing is written.

Alternate embodiments allow the ability to generate a graph of inhibition (given in mm or cm) versus time. This will allow identifying which mixture is more effective in fighting the bacteria/fungi. From the shape of the graph the rate of reaction can also be predicted.

A computerized system may be deployed to report inhibition testing results through a central hub for epedemilogic research and to ascertain whether any disease control procedures are warranted. At the moment it is the physician's or other assigned medical personal responsibility to report any epidemic cases to the government authorities, such as the Center for Disease Control in the United States. Two examples are Chlamydia trachomatis, Genital Infections (Chlamydia trachomatis), Streptococcus pneumoniae, Drug-Resistant Invasive Disease (DRSP), Streptococcus pneumoniae, Drug-Resistant Invasive Disease (DRSP) and many others. This can help map the presence of any pathogen. In addition if the said applied material is a commercial drug it is important to continue the monitoring of the effectiveness of the drug at a long term. This embodiment is usually relevant to physicians, nurses and lab managers.

Upon detection of a specific disearc condition from the inhibition testing, the disase reporting protocols include collecting personal information from a patient, downloading the appropriate form from the CDC web site. An example of an appropriate form would be form CDPH 8555 (2/08) for Escherichia coli 0157:H7.

In other embodiments computer executable instructions may be provided for a software agent that provides a surveillance function for diseases having public health implications. The software agent includes instructions to comply with the medical reporting requirements done by doctors, and may include the image processing combined with the information collected in the wizard to perform classification prediction and estimating which strain of bacteria/fungi has been identified. Preferably, the software agent is operable upon consent from the user so that surveillance information can send to the government repository for epidemic related agents. The software agent may be programmable to keep the reporting user anonymous in the form of anonymous information packets conveyed via the Internet.

Applied materials may be dispensed onto the agar surface in Petri dishes 14 in which the agar is configured into the Petrid dishes 14 as multi-chambered susceptibility plates. The applied materials then are dispensed or made available to the microorganism and the confluent lawns become exposed to a variety of concentrations, or as a gradient of each anti-microbial agent. The plates are then placed in the incubators shown in FIGS. 1, and 6A-B and 7A-C shown below wherein either external or internal cameras monitors and measures the growth (or lack thereof) of the microorganisms.

FIG. 3 is a schematic depiction of preparing a gradient streak of an essential oil or other applied material composition. To a well uniformly inoculated agar surface of a Petri dish 14, a drop 116A having a concentrated essential oil solution 36 is applied. Adjacent to the drop 116A is a diluent drop 1 16B having either no essential oil, the same essential oil at a lower concentration than the essential oil contained in drop 116A, or a different essential oil. When applied, the perimeters of the drop 116A and 116B do not touch. An applicator stick, preferably sterile, migrates through the drop 116A, then through and past the drop 116B along the direction of the indicated arrow in a linear fashion. Drop 116B progressively mixes with or dilutes or merges with drop 116A to render a diminishing streak 116C where the darkest pigment represents the maximal concentration of the essential oil originally residing in drop 116A, and the lightest pigment represents the least concentration of the essential oil originally residing in drop 116A, thus providing substantially continuous gradient. When the drop 116B is a buffer or saline, and the essential oil in drop 116A exhibit substantially inhibitory action to a given bacterial, fungal, or clinical inoculum, a pie slice or wedge shape is presented with the widest portion of the wedge appearing near the most concentrated region of the streak 116C. The gradient produced pie shape inhibition zone helps determine a minimum inhibition concentration (MIC) of a tested essential oil composition.

Depending on the composition and concentration of any essential oil contained in the diluents of drop 116B, the size and shape of the inhibitory zone or pattern can vary with the oil or oil(s) concentration, composition, and incubation times. Analysis of inhibition zones sizes and/or shapes appearing within the acquired digital images can provide predictive therapeutic information of various essential oils, essential oil combinations, and concentrations thereto for treating a given clinical bacterial and/or fungal infection being experienced by the patient user.

FIG. 4 schematically depicts an alternate method to develop a gradient streak or applied discrete spots with self-registration of the applied materials 32 thereon. In this method a linear gradient utilizes gradient concentration such as e-test from Abbiodisk that offers advantages over the drop manipulation methods. It provides for a more controllable and less laborious alternative to the double drop streak method. The gradient is represented by a sector shape two dimensional L-shaped strip 130 having a vertical sector or member and a horizontal sector or member. The whole strip may be fabricated on a matrix (for example, a gore-tex fabric having a transparent appearance). The gore-tex fabric provides structural strength to the strip. Essential oil formulations may be pre-soaked or printed onto the gore-tex strip using an ink-jet printer ontop of the srtip to ensure fixing the drops/lines are accurately in place. The card may have a have a unique shape, i.e. chiral in 2D or other assymetric strip e.g. an “L” or and “F” shape but not symetric shapes similar to the letters A, B, C, E, T, H, I, V, X or O shapes. The asymmetric shape, here depicted as letter “L” serves to aid in the self-registering for applied materials 32 distributed either as discrete spots at application loci A, B, or C in the vertical member of the L-shaped strip 130 or as discrete spots at application loci D, E, F, H along the horizontal member of the L-shaped strip 130. Similarly, the applied materials 32 may be distributed as streaks along the lines of either the horizonal or vertical member of the L-shaped strips. The assymetry of the L-shaped 130 or F-shaped strips ensures easy identification of the applied material's 32 locations to the L-shaped or F-shaped strips.

Another advantage conferred by the two-dimensional L-shaped or F-shaped strips is that of the strips being quick-devolving strip and that the user does not need to deal with the inaccuracies of poised by the dropper bottles 32 in spotting drops of applied materials onto the agar containing Petri Dish 14. The strips do not require the manual skills for droplet manipulation and so the challenges thereto are lessened by utilization of the strips. The strips also prevent mixing unless they are deliberately folded or manually manipulated to touch. The strip are kept in a sterile environment at a temperature to order to maintain integrity of the strip. The advantage of the structural matrix is that the essential oils/hydrosols/herbs and other applied materials can be absorbed to them and ensure their location. Higher temperatures can cause their diffusion from their strip locations faster than lower temperatures. Similarly, the elements in the matrix/strip can be inert lines that ensure that the essential oils/hydrosol and other applied materials will not mix (or move too much) even if they migrate due to thermal diffusion conditions.

The gradient L-shaped strip 130 is represented by a sector shape. The whole strip may be fabricated on a matrix (for example, a Gore-tex® fabric having a transparent appearance). The Gore-tex® fabric provides structural strength to the strip. Essential oil formulations may be pre-soaked or printed onto the gradient strip 130 using an ink-jet printer ontop of the strip to ensure fixing the drops/lines are accurately in place. The advantage of the quick-devolving strip are that the user does not need to deal with the inaccuracies of poised by the dropper bottles 32 in spotting drops onto the agar containing Petri dish 14. The strips do not require the manual skills for droplet manipulation and so the challenges thereto are lessened by utilization of the strips. The strips also prevent mixing unless they are deliberately folded or manually manipulated to touch. The strip are kept in a sterile environment in a specific temperature in order to maintain the integrity of the strip. The advantage of the structural matrix is that the essential oils/hydrosols can be absorbed to them and ensure their location, although high temperatures will cause some diffussion. Similarly, the elements in the matrix/strip can be inert lines that ensure that the essential oils/hydrosol will not mix (or move too much) even if they migrate due to thermal diffusion conditions.

FIG. 6 depicts a series of one-dimensional fabric-based gradient strips. The gradient strips 140, 142, and 144 have respectively compositions A, B, and C of applied materials 32 along the respective lines shown therein. Essential oils or other applied materials 32 are distributed along the lines are similarly applied by inkjet printing as described for FIG. 5. Alternate embodiments of the strips depicted in FIGS. 5 and 6 may also be constructed with a fast or rapid dissolving substrate that is fabricated with hydrophobic threads to hold the essential oils, herbs, antibacterials, or the hydrosols.

Alternate embodiments of the incubator 50 may include being equipped with a UV lamp to insure that there is no mold fungus or bacterial residue that can grow before disposing, and in addition or in its place may have a rinsing method. Other embodiments of the personal incubator 50 include numerous compartments or cells where in each cell there can be a different temperature. The timer control unit, the thermocouple, and the UV lamp ensure incubation adaptability to culture the fungal and/or bacterial content within diverse patient inoculum samples and subsequently provide a decontamination means of the Personal incubator 50 with the UV light or rinsing method to lessen the spread of infectious agent contamination.

FIGS. 6A-B schematically depicts perspective and exploded views of an ambient temperature incubator 150. The ambient temperature incubator 150 is shown having a transparent cover 154 that can rest upon and sealably attaché with chamber box 158. The chamber box 158 may have transparent sides or faces or non-transparent sides or faces. A filter housing 164 receives replaceable filters to block any leakage of toxins (microbes and/or gas or aerosol) from the upside down Petri dishes 14. Air vents 168 allow exchange of outside air that is filtered as the outside air enters the interior of the chamber box 158 that houses the three Petri dishes 14 placed upside down, with or without their lids. The ambient temperature incubator 150 operates in room temperature, thus requiring that the operator place the incubator 150 at a location having a temperature suitable for microbial growth. A typical optimal growth temperature for bacteria is 37 C and fungi us 25 C. Upon disposing the user can use a UV lamp (not shown) for few minutes when opening the incubator or spray the interior of the chamber box 158 with a disinfectant such as an antibacterial or anti fungal liquid. The transparent top 154 allows the user to lean over the incubator to monitor or take pictures or video (with a hand held camera) of the changes occurring in the Petri dishes 14. The transparent cover 154 may be connected with the chamber box 158 with hinges similar to those depicted in incubator 50 of FIG. 1.

The replaceable filters inserted into the filter housing 164 may include any one of the following types, as long as they block the leakage of toxins or pathogen outside the box. 1) a high efficiency particulate air (HEPA) filter may remove at least 99.97% of airborne particles 0.3 micrometers in diameter, thus making it safe to use outside of a laboratory setting. HEPA filter detail are disclosed in U.S. Pat. No. 6,428,610 to Tsai, et al. 2) A filter made of non-woven form with anti bacterial, anti viral and anti fungal material made of tea extract as disclosed in U.S. Pat. No. 5,888,527 to Nasimoto et al. is an alternative to a high efficiency particulate air (HEPA) filter. 3) A filter made of thermoplastic resin as disclosed in U.S. Pat. No. 6,165,243 given to Byassee. All patent references above incorporated by reference in their entirety.

The ambient temperature incubator 150 provides for the individual to utilize the incubator 150 (with one of the filters as mentioned above) to develop a clearance or inhibition zone around an applied material 32 (essential oil, hydrosol, tincture, silver ion, antibiotic etc) distributed onto the agar surfaces of Petri dishes 14. The inhibition or clearance zones may be photographed for latter measurement. A ruler may be placed at the level of the inhibition zones to provide a means to calculate a magnification factor of the inhibition zones images to that of the inhibition zone object. Analysis of the clearance zones per applied material 32 provides the basis to aid the individual in accessing the therapeutic potential of the at least one applied material.

An alternate embodiment of the incubator 150 provides for the swabbed plates 14 being placed in the incubator 150 and against a ruler ledge (referred to as an “object ruler” at the height of the inhibition zone. Then the user can take pictures of the agar plates 14 through the transparent cover 154 using a user hand held digital camera, either all three at the same time, or plate-by-plate, in which the ruler ledge or object ruler is in the camera view and captured with the agar plate image. Having the built in object ruler adjacent to the plates allows for assessing a calibration factor (or magnification factor) to be calculated so that inhibition zones can be measured on the computer screen with a computer generated ruler (denoted as an “image ruler”) and any change in magnification caused by the camera and/or the computer screen projection can be ascertained by the measuring on the computer screen an increment of the object ruler with the “image ruler” so that the actual magnification correction factor is determined by the quotient of the “image ruler” measurements of object ruler increment/“object ruler” increment. Thus a 10 mm object ruler increment might be measured as 50 mm image ruler increment on the computer screen, so that the magnification factor is 5. Thus if a 20 mm inhibition zone is measured on the computer screen via the image ruler, the actual distance that would be measured at the agar plate would be 4 mm (image distance/magnification factor=20 mm/5=4 mm). From these distances relative assessments can be made or quantitative assessments when undertaken in a controlled manner.

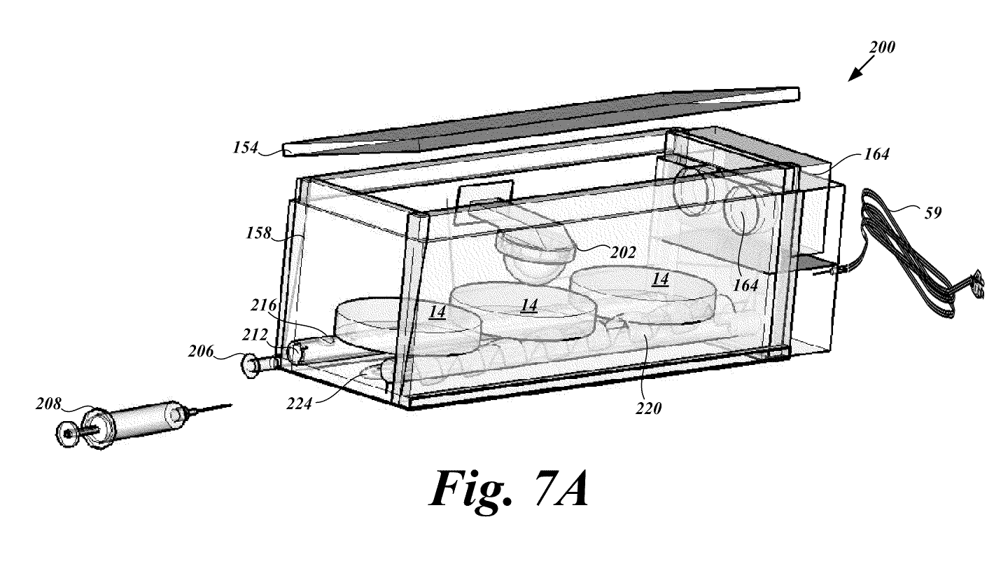

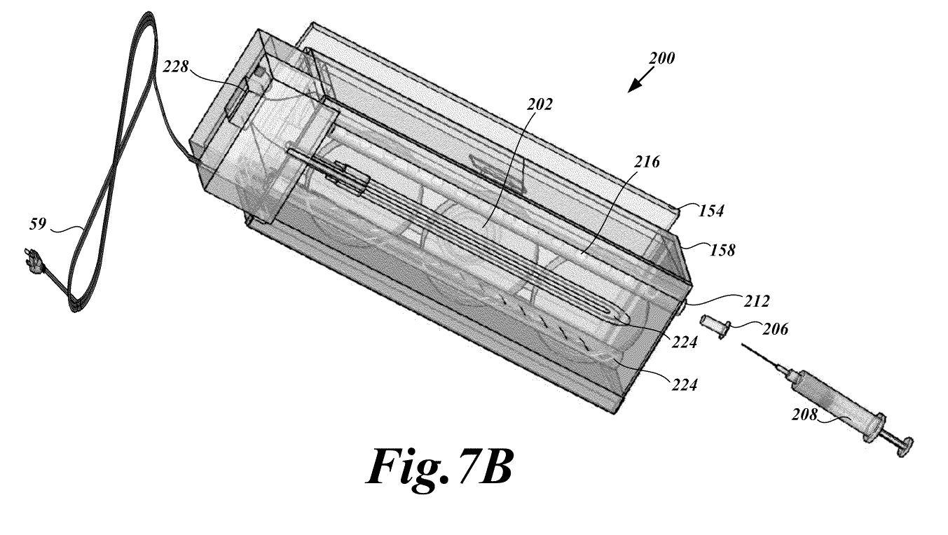

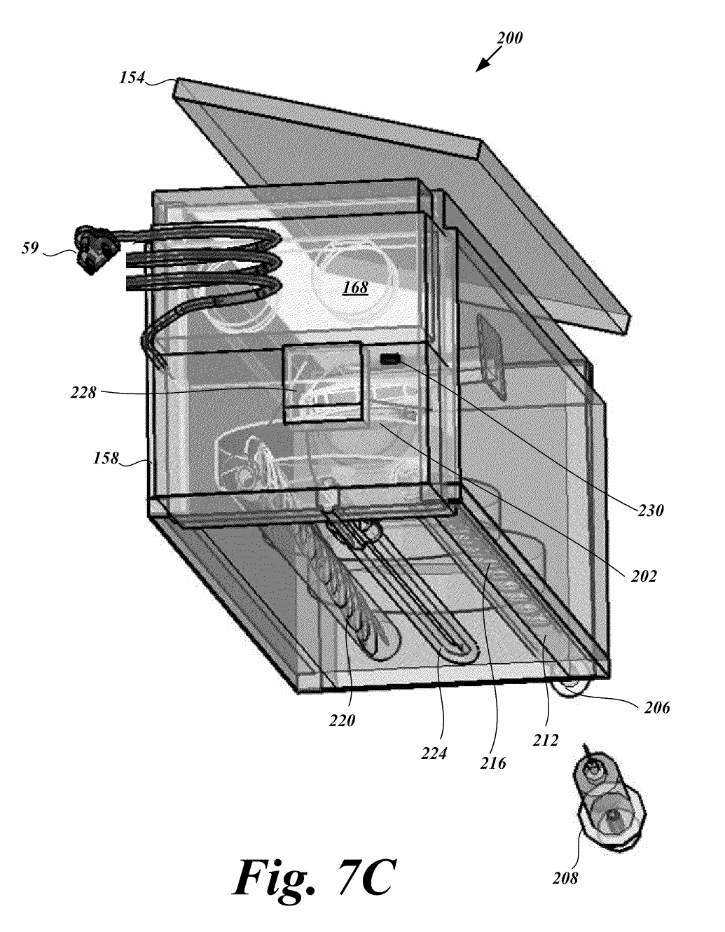

FIGS. 7A-C schematically depicts in side, bottom and end perspective and exploded views of a sanitizable temperature controlled incubator 200. The sanitizable temperature controlled incubator 200 includes the ambient temperature incubator's 150 transparent cover 154, the box chamber 158, the filter housing 164 for receiving insertable filters, the air exchange vents 164, and personal incubators 50 power supply cord 59. Also included in the incubator 200 is camera 202 configured to acquire images of the clearance zones within the interior of the sanitizable temperature controlled incubator includes a camera configured to acquire images of the clearance zones within the interior of the sanitizable temperature controlled incubator temperature controlled and sanitizable personal incubator. The sanitizable personal incubator 200 further includes a removable sealing plug 206 exposing a channel to receive a syringe 208 injection into a punctured pipe or wash tube 212 having orifices 216 aimed to provide plumes of disinfectant solution to make contact with the Agar surface microbial growth of upside down and lidless Petri dishes 14. Also included is heater coil 220, a temperature and controller box 228 in electrical communication with the heater coil 220, a USB port 230 in signal communication with the digital camera 202. The display and control unit 228 may include a digital display and be configured to send images acquired from the camera 202 via the USB port 230 for image processing utilize software executable instructions retrievable from a PC or on a microcontroller (not shown). The PC also is configured with reporting capability to the user and through the Internet or other network in communication with a disease reporting or other public health entity. The reporting capability provides for the user to enter the subject information and the said software has means of reporting the finding to central disease control (CDC), USDA or any other public heath entity.

Other embodiments of the incubator may employ the bacteria disinfection component configured to destroy bacteria, fungi, parasites or other biological materials having a confluent growth pattern with disinfection solutions before disposing of the Petri dish or with a UV lamp. The

The UV lamp 224 is mercury based and configured to emit a wavelength within a range of 185 to 254 nanometers to foster killing of live cultures on the agar surfaces before disposing the petri dish 14. An example is 15 W 185 nanometer lamp 18″ length from Philips. The lamp may be encased in a UV-transparent water proof shield to protect the lamp from anything dripping on it. The UV lamp 224 receives electrical power from via the power supply cord 59 and transformer based circuitry (not shown). Alternatives to the UV lamp 224 include a UV LED lamp.

In case where the microbe is a hazardous or unknown pathogen where the petri dish needed to be placed facing up the UV disinfecting method is the preferred method as long as the dish glass or plastic is transparent to UV at the given wavelength range.

An alternative embodiment to the UV lamp 224, is rinsing with antimicrobial material. the punctured pipe 212 with an inlet on one side of the incubator can be used. It should be placed in the lower corner of the incubator and sealed at all time. The holes or orifices 216 in the pipe may be about 45 degree tilted toward the upside down lidless Petri dishes 14 to ensure complete coverage of the dish(s) 14. There may be more holes 216 in the pipe as the distance from the wall is increasing. This aperture 216 distribution provides that the same amount of liquid will be applied to the near, middle, and far Petri dishes in relation to the distance from the inlet. For example next to the inlet we will have one hole 216, in the middle we will have double the number of holes 216 and at the far end we will have triple the number (or size) of the holes 216. The syringe 208 can deliver the disinfectant liquid into the inner chamber of the chamber box 158. To ensure complete coverage the syringe is inserted such that the end of the needle will be close to the holes that will serve mini geysers. The excess disinfect liquid and any dissolved media or sample will be dripping and be collected in the bottom of the incubator. At the end of the disinfection process the user needs to remove the cap that seals the pipe and tilt the whole incubator to drain any remaining liquids.

An example for disinfect are the following: Ethyl alcohol, Hydrogen peroxide, Octyl decyl dimethyl ammonium chloride, among many disinfecting material that can be used. Another example for antibacterial liquid is disclosed in U.S. Pat. No. 6,147,039 to Jacques et al., and the anti-fungal formulation is disclosed in U.S. Pat. Nos. 5,519,059 and 6,048,836 to Romano et al, U.S. Pat. No. 6,346,281 to DeAth. All patents incorporated by reference in their entirety.

If a disinfectant is going to be used, the bottom of the incubator will need to be altered. The corner where the punctured pipe is extruding will be lower than the other three corners. This will allow easy drainage of any material out when we unplug the pipe.

Optimal conditions or acceleration of growth may require temperature control. One case is a heater element including heating control unit and external power source. Another option is to add a cooling/heating element such as Peltier.

Other embodiment will be other settings mentioned above and a heating element, sensor, control and external power source. Other embodiment will be any setting mentioned above plus A circuit for cooling/heating such as Peltier.

If a rinsing pipe and/or UV lamp and/or heater is part of the incubator a net or a shelf can be placed on top of these part to allow placement of different size Petri dishes. The mentioned above is another embodiment. The requirement is that the shelf or net will allow the disinfectant material to cover the dish(s) area.

Other embodiment is made for safety reasons. A locking mechanism controlled by pressure sensor and a sealing mechanism to block any ventilation ports before dismissing the Petri dish may be installed. If a hazardous pathogen or live cells were grown and the UV lamp was used or the antimicrobial material was used to rinse the incubator, this locking mechanism will ensure that no live pathogen will survive. The locking mechanism will ensure that nobody will be able to accidental open the incubator before the pathogen is completely destroyed. For the pressure sensor see for example U.S. Pat. No. 4,152,213 by Ahnell, herein incorporated by reference in its entierty. In addition to other settings the incubator may include a locking mechanism and pressure sensor.

Other embodiment is the following case: if the pathogen is anaerobic CO2 presence may be needed to provide optimal conditions as disclosed in U.S. Pat. No. 3,591,461 to Bazil et al. and Tena at el. Emerg Infect Dis. July2008; 14(7): 1163-1164, herein incorporated by reference in their entirety. In addition to other settings the incubator has a gas intake and distribution will be added.

In addition to the previous embodiment an additional embodiment will be the addition of gas sensors. In case an external gas (mixture) is provided, a set of gas sensor can be added to provide feedback for the incoming mixture. Examples are: CO2, N2, O2.

For faster results the incubator may be placed on a shaker. Other embodiment is adding a shaker. For example in addition to other settings the incubator has four legs that at given moment the legs get shorter/longer thus tilting the dish (not shown).

For ease of control a timer may be controlling the incubator and alerting the user upon completion.

Alternate embodiments of the incubator include a personal incubator, and an optional camera to record the image of the inhibition zone. Other embodiments provide for a disinfection device to inactivate the pathogen containing agar plates before disposing. Yet other embodiments of the aromatogram system provide for software analysis of the inhibition zones to help the at home user predict the therapeutic potential of at least one essential oil composition and/or concentration.

The digital camera 202 may take pictures of the process or the final status of the dish. The information in the picture or movie can be stored and process locally or transferred to a personal computer (PC) via wired communication, for example using a universal serial bus (USB) or via wireless communication for analysis. In addition to other settings the incubator may include a digital camera, a USB port and/or wireless connection and/or card writer to handle memory cards such as MMC, MS, MS pro SD, SD pro or xD or any other card type. The camera housing is mounted on one wall. In the camera itself is mounted inside the housing to allow the camera to face the Petri dish(s). Illumination of the inhibition zones if ambient illumination through the transparent cover 154 is not enough may be achieved by use of a white light emitting diodes (LED) can be used when the picture or video are being taken. As disclosed in U.S. Pat. No. 6,696,269 to Newell, incorporated by reference in its entirety.

Other embodiments for the sanitizable temperature controlled incubator 200 would include a rotatable platter having cutouts to load upside down lidless agar plates 14. The platter could then rotate the agar plates to be photographed to beneath the camera 202, then rotated to be exposed to the underlying UV light source, then rotated to receive a disinfection solution. The incubator 200 may also include a timer to alert the user at a said time or shut off the operation of the incubator. The camera 202 is configured to acquire images of the clearance zones within the interior of the sanitizable temperature controlled incubator. Yet other embodiments of the incubator 200 provide a pressure sensor, a lock and a sealing mechanism for the ventilation. Moreover, the camera 202 includes the means of transferring the image through USB connection via USB port 230 or collected in a memory cards such as MMC, MS, MS pro SD, SD pro or xD or any other card type. A power in form or external power cord or batteries (not shown) may be used as needed. The camera 202 may include one or more color and/or gray scale imaging devices having a charge coupled device (CCD) linear array scanner, a CCD line-scan camera, a still CCD 2D array camera, a motion video CCD 2D array camera or a laser scanning camera. The camera 202 may be re-positionable to capture images above and beneath the Petri dishes 14.

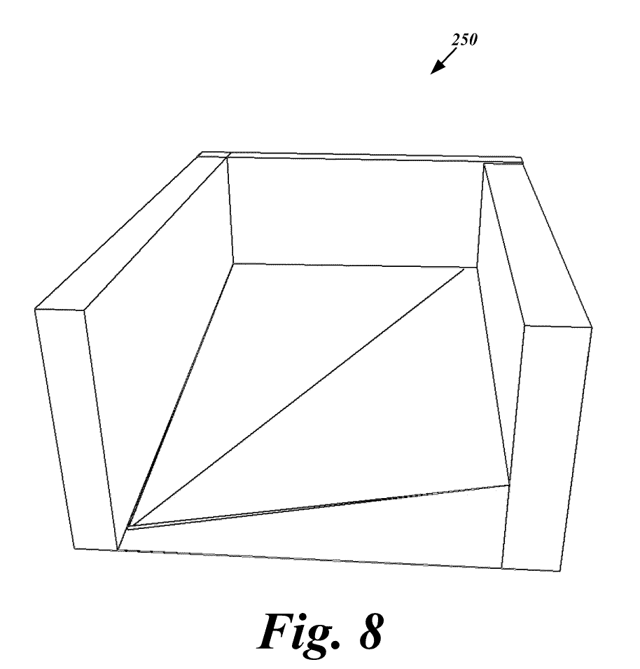

FIG. 8 depicts an alternate embodiment of the personal incubator of FIGS. 6A-C having a tiltable base The tilt allows drainage of any excess liquids. The sealing plug when removed allows removal of liquids (not shown).

While the preferred embodiment of the invention has been illustrated and described, as noted above, many changes can be made without departing from the spirit and scope of the invention. For example, individual Petri dishes 14 may be configured into split plates or be configured in microliter plate formats having a panel of wells with growth agar in place. The microliter plates may include more than two wells, and commonly up to but not limited to 24 wells per plate. Instead, the invention should be determined entirely by reference to the claims that follow.

The embodiments of the invention in which an exclusive property or privilege is claimed are defined as follows:

1. A system to conduct inhibition diagnostic testing by a user comprising:
a kit having at least one Petri dish, at least one sterile swab, at least one media packet, and at least one applied material composition;
an incubator to develop a clearance zone around an applied essential oil, the incubator having a digital camera to acquire at least one image of the clearance zone; and
means for analysis of the at least one image of the clearance zone,
wherein information from the analysis aids the user in accessing therapeutic potential of the at least one oil composition.

2. The system of claim 1, wherein the at least one applied material composition includes essential oils comprising carvacrol, geraniol, linalool, terpinen-4-ol, alpha-terpineol, alpha-pinene, 1,8, cineole, citral, citronellal, thymol, eugenol, mentha piperita, melaleuca alternifolia, lavandula angustifolia, ocimum basilicum, origamum vulgare, satureja montana, thymus vulgaris, backhousia citriodora, cymbopogon citratus, lavandula officinalis, melaleuca cajuputi, mellisa officinalis, ocimum gratissimum, origanum vulgare ssp. Hirtum, pelargonium graveolens, rosmarinus officinalis, syzygium aromaticum, cymbopogan citratus, cymbopogon martinii, melaleuca cajuputi, ocimum gratisimum, pelargonium graveolens, piper nigrum, pogostemon patchouli, santalum album, syzyfium aromatium, vetiveria zizanioides, thypus capitatus, backhusia citriodora, origanum vulgare, lavandula latifolia, pogostemon patchouli, sentalum album, rosmarinus officinalis, lavandula angustifolia, melaleuca alternifolia, and essential oil nano-emulsions.

3. The system of claim 1, wherein the at least one applied material composition includes herbs, antibiotics, antifungals, silver compounds, and nano emulsions.

4. The system of claim 1, wherein the incubator is an ambient temperature incubator having a transparent top configured to acquire images of the clearance zones.

5. The system of claim 1, wherein the incubator is a sanitizable temperature controllable incubator having a transparent top configured to acquire images of the clearance zones visible through the transparent top.

6. The system of claim 5, wherein the sanitizable temperature controlled incubator includes a camera configured to acquire images of the clearance zones within the interior of the sanitizable temperature controlled incubator.

7. The system of claim 5, wherein the sanitizable temperature controlled incubator includes a disinfection hydraulic member configured to deliver disinfection solutions to the Petri dish having biological growth.

8. An inhibition diagnostic kit comprising:
at least one petri dish;
at least one media preparation;
at least one sterile swab; and
at least one applied material.

9. The diagnostic kit of claim 8, wherein at least one applied material includes essential oils comprising carvacrol, geraniol, linalool, terpinen-4-ol, alpha-terpineol, alpha-pinene, 1,8, cineole, citral, citronellal, thymol, eugenol, mentha piperita, melaleuca alternifolia, lavandula angustifolia, ocimum basilicum, origamum vulgare, satureja montana, thymus vulgaris, backhousia citriodora, cymbopogon citratus, lavandula officinalis, melaleuca cajuputi, mellisa officinalis, ocimum gratissimum, origanum vulgare ssp. Hirtum, pelargonium graveolens, rosmarinus officinalis, syzygium aromaticum, cymbopogan citratus, cymbopogon martinii, melaleuca cajuputi, ocimum gratisimum, pelargonium graveolens, piper nigrum, pogostemon patchouli, santalum album, syzyfium aromatium, vetiveria zizanioides, thypus capitatus, backhusia citriodora, origanum vulgare, lavandula latifolia, pogostemon patchouli, sentalum album, rosmarinus officinalis, lavandula angustifolia, melaleuca alternifolia, and essential oil nano-emulsions.

10. The diagnostic kit of claim 8, wherein the at least one applied material composition includes herbs, antibiotics, antifungals, silver compounds, and nano emulsions.

11. A method to conduct aromatogram diagnostic testing by a user comprising:
preparing a clinical specimen inoculum from the user;
preparing at least one petri dish medium for receiving the clinical specimen inoculum;
applying at least one applied material to the at least one petri dish having the clinical specimen inoculum;
acquiring an image of the inhibition zone around the at least one essential oil; and
ascertaining therapeutic potential of the at least one applied material based upon the analysis of the inhibition zone.

12. The method of claim 11, wherein applying the at least one applied material includes essential oils comprising carvacrol, geraniol, linalool, terpinen-4-ol, alpha-terpineol, alpha-pinene, 1,8, cineole, citral, citronellal, thymol, eugenol, mentha piperita, melaleuca alternifolia, lavandula angustifolia, ocimum basilicum, origamum vulgare, satureja montana, thymus vulgaris, backhousia citriodora, cymbopogon citratus, lavandula officinalis, melaleuca cajuputi, mellisa officinalis, ocimum gratissimum, origanum vulgare ssp. Hirtum, pelargonium graveolens, rosmarinus officinalis, syzygium aromaticum, cymbopogan citratus, cymbopogon martinii, melaleuca cajuputi, ocimum gratisimum, pelargonium graveolens, piper nigrum, pogostemon patchouli, santalum album, syzyfium aromatium, vetiveria zizanioides, thypus capitatus, backhusia citriodora, origanum vulgare, lavandula latifolia, pogostemon patchouli, sentalum album, rosmarinus officinalis, lavandula angustifolia, melaleuca alternifolia, and essential oil nano-emulsions.

13. The method of claim 10, wherein the at least one applied material composition includes herbs, antibiotics, antifungals, silver compounds, and nano emulsions.
